SEQUENCE LISTING (18 sequences)

SEQ ID NO: 1 - protein, 6 aa, Artificial Sequence
  Heavy chain CDR1
TAYYIH

SEQ ID NO: 2 - protein, 17 aa, Artificial Sequence
  Heavy chain CDR2, H2
YISCYNGATNYNQKFKG

SEQ ID NO: 3 - protein, 17 aa, Artificial Sequence
  Heavy chain CDR2, H7
YISSYNGATNYNQKFKG

SEQ ID NO: 4 - protein, 17 aa, Artificial Sequence
  Heavy chain CDR2 H9
YISVYNGATNYNQKFKG

SEQ ID NO: 5 - protein, 11 aa, Artificial Sequence
  Heavy chain CDR3
RDYDYDVGMDY

SEQ ID NO: 6 - protein, 119 aa, Artificial Sequence
  Heavy chain variable region without signal sequence, H7
QVQLVQSGAEVKKPGASVKISCKASGYSFTAYYIHWVKQAPGQGLEWIGYISSYNGATNYNQKFKGRVTFTTDTSTSTAYMELRSLRSDDTAVYYCARDYDYDVGMDYWGQGTLVTVSS

SEQ ID NO: 7 - protein, 15 aa, Artificial Sequence
  Light chain CDR1
RASESVDNYGISFMK

SEQ ID NO: 8 - protein, 7 aa, Artificial Sequence
  Light chain CDR2
AASNQGS

SEQ ID NO: 9 - protein, 12 aa, Artificial Sequence
  Light chain CDR3
QQSKEVPWTFGG

SEQ ID NO: 10 - protein, 111 aa, Artificial Sequence
  Light chain variable region without signal sequence
DIVMTQSPDSLAVSLGERATISCRASESVDNYGISFMKWFQQKPGQPPKLLIYAASNQGSGVPDRFSGSGSGTDFTLTISSLQAEDVAVYYCQQSKEVPWTFGGGTKVEIK

SEQ ID NO: 11 - protein, 191 aa, Artificial Sequence
  Amino acids 27-217 of Human DLL4 Extracellular Domain (without putative signal sequence)
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYC

SEQ ID NO: 12 - protein, 119 aa, Artificial Sequence
  Heavy chain variable region without signal sequence, H2
QVQLVQSGAEVKKPGASVKISCKASGYSFTAYYIHWVKQAPGQGLEWIGYISCYNGATNYNQKFKGRVTFTTDTSTSTAYMELRSLRSDDTAVYYCARDYDYDVGMDYWGQGTLVTVSS

SEQ ID NO: 13 - protein, 119 aa, Artificial Sequence
  Heavy chain variable region without signal sequence, H9
QVQLVQSGAEVKKPGASVKISCKASGYSFTAYYIHWVKQAPGQGLEWIGYISVYNGATNYNQKFKGRVTFTTDTSTSTAYMELRSLRSDDTAVYYCARDYDYDVGMDYWGQGTLVTVSS

SEQ ID NO: 14 - protein, 189 aa, Homo sapiens [sequence omitted]

SEQ ID NO: 15 - protein, 496 aa, Homo sapiens
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVG

SEQ ID NO: 16 - protein, 154 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQN

SEQ ID NO: 17 - protein, 63 aa, Homo sapiens
WLLDEQTSTLTRLRYSYRVICSDNYYGDNCSRLCKKRNDHFGHYVCQPDGNLSCLPGWTGEYC

SEQ ID NO: 18 - protein, 7 aa, Artificial Sequence
  FLAG tag
DYKDDDK

(19) United States
(12) Patent Application Publication
(10) Pub. No.: US 2016/0243223 A1
(43) Pub. Date: Aug. 25, 2016

(54) METHODS FOR TREATING CANCERS COMPRISING K-RAS MUTATIONS

(71) Applicant: OncoMed Pharmaceuticals, Inc., Redwood City, CA (US)

(72) Inventors: Timothy C. HOEY, Hillsborough, CA (US); Wan-Ching Yen, Foster City, CA (US); Marcus M. Fischer, Sunnyvale, CA (US)

(73) Assignee: OncoMed Pharmaceuticals, Inc.

(21) Appl. No.: 14/962,127

(22) Filed: Dec. 8, 2015

Related U.S. Application Data
(63) Continuation of application No. 12/957,741, filed on Dec. 1, 2010, now abandoned.
(60) Provisional application No. 61/265,559, filed on Dec. 1, 2009.

(51) Int. Cl. A61K 39/395; A61K 45/06

(52) U.S. Cl. CPC A61K 39/3955; A61K 45/06; A61K 2039/505

(57) ABSTRACT

Methods of inhibiting tumor growth, methods of treating cancer, and methods of reducing the frequency of cancer stem cells in a tumor are described. Particularly, the methods are directed to tumors or cancers that comprise a K-ras mutation. The methods described comprise administering a DLL4 antagonist (e.g., an antibody that specifically binds the extracellular domain of human DLL4) to a subject. Related polypeptides and polynucleotides, compositions comprising the DLL4 antagonists, and methods of making the DLL4 antagonists are also described.

### CROSS REFERENCE TO RELATED APPLICATIONS

This application is a continuation of U.S. application Ser. No. 12/957,741, filed Dec. 1, 2010, which claims the benefit of U.S. Provisional Application No. 61/265,559, filed Dec. 1, 2009, all of which are herein incorporated by reference.

### REFERENCE TO SEQUENCE LISTING SUBMITTED ELECTRONICALLY VIA EFS-WEB

The content of the electronically submitted sequence listing (Name: 22930630002sequence_listing_ascii.txt; Size: 16,691 bytes; Date of Creation: Dec. 4, 2015) submitted in this application is incorporated herein by reference in its entirety.

### FIELD OF THE INVENTION

The field of this invention generally relates to antibodies and other agents that bind to DLL4 proteins, as well as methods of using the antibodies or other agents for the treatment of diseases, such as cancer, particularly cancers comprising K-ras mutations.

### BACKGROUND OF THE INVENTION

Cancer is one of the leading causes of death in the developed world, resulting in over 550,000 deaths per year in the United States alone. Almost one and half million people are diagnosed with cancer in the U.S. each year, and currently one in four deaths in the U.S. is due to cancer. (Jemal et al., 2008, Cancer J. Clin. 58:71-96). Although there are many drugs and compounds currently available and in use, these numbers show that a need continues to exist for new therapeutic agents for the treatment of cancer.

Many interlinked signaling pathways play an important role in tumorigenesis via phosphorylation of various proteins and transcription factors that directly control cell growth, differentiation and apoptosis. K-ras, a member of the rat sarcoma virus (ras) gene family of oncogenes, encodes the guanosine diphosphate (GDP)- and guanosine triphosphate (GTP)-binding protein Ras that acts as a self-inactivating intracellular signal transducer. Ras functions as an intermediary downstream of many receptor tyrosine kinases to transmit growth factor signals from the membrane to the MAP kinase cascade. Initiation of MAP signaling ultimately leads to expression of proteins playing roles in cell growth, differentiation, migration and survival. Mutation(s) in K-ras (as well as other genes in the pathway) can result in continuous activity of the Ras-MAPK pathway. (See review Siena et al., 2009, JNCI, 101:1308-1324). Activating mutations in the Ras protein typically occur in residues 12, 13, 59 or 61 and impair the GTPase activity of the molecule, leading to constitutive activation of Ras signaling. Ras mutations are found in approximately one-third of all human cancers and K-ras mutations account for most of the Ras mutations in the majority of human cancers. For example, K-ras mutations are frequent in colon cancer, with approximately 45% of tumors from colon cancer patients containing an activating mutation. In addition, activating K-ras mutations have been found in non-small cell lung cancers at a frequency of approximately 35%.

Antibodies that target the epidermal growth factor receptor (EGFR), cetuximab and panitumumab, have been shown to be effective in the treatment of colon cancer. However, in several retrospective studies, these antibodies have been shown to be ineffective in patients whose tumors comprise activating mutations in the K-ras oncogene (Benvenuti et al. 2007, Cancer Res., 67:2643-2648; Chau and Cunningham, 2009, British J. Cancer, 100:1704-1719). Thus, there is a need for new agents that could provide therapeutic benefit for this large segment of colon cancer patients.

The Notch signaling pathway is a universally conserved signal transduction system. It is involved in cell fate determination during development including embryonic pattern formation and post-embryonic tissue maintenance. In addition, Notch signaling has been identified as a critical factor in the maintenance of hematopoietic stem cells (HSCs).

The Notch pathway has been linked to the pathogenesis of both hematologic and solid tumors and cancers. Numerous cellular functions and microenvironmental cues associated with tumorigenesis have been shown to be modulated by Notch pathway signaling, including cell proliferation, apoptosis, adhesion, and angiogenesis. (Leong et al., 2006, Blood, 107:2223-2233). In addition, Notch receptors and/or Notch ligands have been shown to play potential oncogenic roles in a number of human cancers, including acute myelogenous leukemia, B cell chronic lymphocytic leukemia, Hodgkin lymphoma, multiple myeloma, T cell acute lymphoblastic leukemia, brain cancer, breast cancer, cervical cancer, colon cancer, lung cancer, pancreatic cancer, prostate cancer and skin cancer. (Leong et al., 2006, Blood, 107:2223-2233). Thus, the Notch pathway has been identified as a potential target for cancer therapy.

Previous studies demonstrated that antibodies to the human Notch ligand DLL4 (Delta-like ligand 4) can decrease the percentage of cancer stem cells in some xenograft tumors. In addition, antibodies to mouse DLL4 were shown to result in hyperproliferation of tumor vasculature. (Hoey et al., 2009, Cell Stem Cell, 5:168-177). These findings suggest that targeting the Notch pathway, for example with DLL4 antagonists, could help eliminate not only the majority of non-tumorigenic cancer cells, but the tumorigenic cancer stem cells responsible for the formation and recurrence of solid tumors.

### SUMMARY OF THE INVENTION

The present invention provides methods of inhibiting growth of a tumor comprising administering a therapeutically effective amount of a DLL4 antagonist to a human subject, wherein the tumor comprises a K-ras mutation. In some embodiments, the DLL4 antagonist is an antibody that specifically binds the extracellular domain of human DLL4. In some embodiments, the tumor is a colorectal tumor, a lung tumor, a pancreatic tumor, a liver tumor or multiple myeloma.

In another aspect, the invention provides methods of inhibiting growth of a tumor comprising administering a therapeutically effective amount of a DLL4 antagonist to a human subject, wherein the tumor is substantially non-responsive to at least one EGFR inhibitor. In some embodiments, the DLL4 antagonist is an antibody that specifically binds the extracellular domain of human DLL4. In some embodiments, the tumor that is substantially non-responsive to at least one EGFR inhibitor comprises a K-ras mutation. In some embodiments, the tumor is a colorectal tumor, a lung tumor, a pancreatic tumor, a liver tumor or multiple myeloma.

In one aspect, the invention provides methods of treating cancer in a human subject, comprising (a) determining that the subject's cancer comprises a K-ras mutation, and (b) administering to the subject a therapeutically effective amount of a DLL4 antagonist. In some embodiments, the cancer is colorectal cancer, lung cancer, pancreatic cancer, liver cancer or multiple myeloma.

In another aspect, the invention provides methods of treating cancer in a human subject, comprising (a) selecting a subject for treatment based, at least in part, on the subject having a cancer that comprises a K-ras mutation, and (b) administering to the subject a therapeutically effective amount of a DLL4 antagonist. In some embodiments, the cancer is colorectal cancer, lung cancer, pancreatic cancer, liver cancer or multiple myeloma.

In another aspect, the invention provides methods of treating cancer in a human subject, comprising (a) identifying a subject that has a cancer comprising a K-ras mutation, and (b) administering to the subject a therapeutically effective amount of a DLL4 antagonist. In some embodiments, the cancer is colorectal cancer, lung cancer, pancreatic cancer, liver cancer or multiple myeloma.

In another aspect, the invention provides methods of treating cancer in a human subject, comprising (a) determining that the subject's cancer is substantially non-responsive to at least one EGFR inhibitor, and (b) administering to the subject a therapeutically effective amount of a DLL4 antagonist. In some embodiments, the cancer that is substantially non-responsive to at least one EGFR inhibitor comprises a K-ras mutation. In some embodiments, the cancer is colorectal cancer, lung cancer, pancreatic cancer, liver cancer or multiple myeloma.

In another aspect, the invention provides methods of treating cancer in a human subject, comprising (a) selecting a subject for treatment based, at least in part, on the subject having a cancer that is substantially non-responsive to at least one EGFR inhibitor, and (b) administering to the subject a therapeutically effective amount of a DLL4 antagonist. In some embodiments, the cancer that is substantially non-responsive to at least one EGFR inhibitor comprises a K-ras mutation. In some embodiments, the cancer is colorectal cancer, lung cancer, pancreatic cancer, liver cancer or multiple myeloma.

In another aspect, the invention provides methods of treating cancer in a human subject, comprising (a) identifying a subject that has a cancer that is substantially non-responsive to at least one EGFR inhibitor, and (b) administering to the subject a therapeutically effective amount of a DLL4 antagonist. In some embodiments, the cancer that is substantially non-responsive to at least one EGFR inhibitor comprises a K-ras mutation. In some embodiments, the cancer is colorectal cancer, lung cancer, pancreatic cancer, liver cancer or multiple myeloma.

In another aspect, the invention provides methods of selecting a human subject for treatment with a DLL4 antagonist, comprising determining if the subject has (a) a cancer comprising a K-ras mutation, or (b) a cancer that is substantially non-responsive to at least one EGFR inhibitor, wherein if the subject has (a) and/or (b), the subject is selected for treatment with a DLL4 antagonist.

In certain embodiments of each of the aforementioned aspects, as well as other aspects and embodiments described elsewhere herein, the K-ras mutation is detected in a sample by methods known to those skilled in the art, such as PCR-based assays or direct nucleotide sequencing. In some embodiments, the sample is a fresh tumor sample, a frozen tumor sample, or a formalin-fixed paraffin-embedded sample.

In certain embodiments of each of the aforementioned aspects, as well as other aspects and embodiments described elsewhere herein, the K-ras mutation is an activating mutation. In some embodiments, the tumor or cancer comprises more than one K-ras mutation. In some embodiments, the K-ras mutation is a mutation in codon 12, 13, 59 and/or 61. In some embodiments, the EGFR inhibitor is a small molecule compound or an antibody. In some embodiments, the EGFR inhibitor is the anti-EGFR antibody cetuximab or panitumumab. In some embodiments, the EGFR inhibitor is erlotinib or gefitinib.

In certain embodiments of each of the aforementioned aspects, as well as other aspects and embodiments described elsewhere herein, the DLL4 antagonist is an antibody that specifically binds human DLL4. In some embodiments, the antibody specifically binds an epitope comprising amino acids within the N-terminal region of human DLL4 (SEQ ID NO:16).

In some embodiments, the DLL4 antagonist is an antibody comprising: (a) a heavy chain CDR1 comprising TAYYIH (SEQ ID NO:1), a heavy chain CDR2 comprising YISCYNGATNYNQKFKG (SEQ ID NO:2), YISSYNGATNYNQKFKG (SEQ ID NO:3), or YISVYNGATNYNQKFKG (SEQ ID NO:4), and a heavy chain CDR3 comprising RDYDYDVGMDY (SEQ ID NO:5); and/or (b) a light chain CDR1 comprising RASESVDNYGISFMK (SEQ ID NO:7), a light chain CDR2 comprising AASNQGS (SEQ ID NO:8), and a light chain CDR3 comprising QQSKEVPWTFGG (SEQ ID NO:9). In some embodiments, the DLL4 antagonist is an antibody comprising: (a) a heavy chain CDR1 comprising TAYYIH (SEQ ID NO:1), a heavy chain CDR2 comprising YISSYNGATNYNQKFKG (SEQ ID NO:3), and a heavy chain CDR3 comprising RDYDYDVGMDY (SEQ ID NO:5); and (b) a light chain CDR1 comprising RASESVDNYGISFMK (SEQ ID NO:7), a light chain CDR2 comprising AASNQGS (SEQ ID NO:8), and a light chain CDR3 comprising QQSKEVPWTFGG (SEQ ID NO:9).

In certain embodiments of each of the aforementioned aspects, as well as other aspects and embodiments described elsewhere herein, the DLL4 antagonist is an antibody comprising (a) a heavy chain variable region having at least about 90%, at least about 95% or 100% sequence identity to SEQ ID NO:6, SEQ ID NO:12 or SEQ ID NO:13; and/or (b) a light chain variable region having at least about 90%, at least about 95% or 100% sequence identity to SEQ ID NO:10. In some embodiments, the DLL4 antagonist is antibody 21M18, 21M18 H7L2 or 21M18 H9L2. In some embodiments, the DLL4 antagonist is the antibody encoded by the plasmid having ATCC deposit no. PTA-8425 which was deposited with the ATCC under the conditions of the Budapest Treaty on May 10, 2007. In some embodiments, the DLL4 antagonist is the antibody encoded by the plasmid having ATCC deposit no. PTA-8427 which was deposited with the ATCC under the conditions of the Budapest Treaty on May 10, 2007. In some embodiments, the DLL4 antagonist is the antibody produced by the hybridoma having ATCC deposit no. PTA-8670 which was deposited with the ATCC under the conditions of the Budapest Treaty on Sep. 28, 2007.

In certain embodiments of each of the aforementioned aspects, as well as other aspects and embodiments described elsewhere herein, the DLL4 antagonist is a recombinant antibody. In some embodiments, the antibody is a monoclonal antibody, a chimeric antibody, a humanized antibody, or a human antibody. In some embodiments, the antibody is an antibody fragment. In certain embodiments, the antibody or antibody fragment is monovalent, monospecific, bivalent, bispecific, or multispecific. In certain embodiments, the antibody is isolated. In other embodiments, the antibody is substantially pure.

In certain embodiments of each of the aforementioned aspects, as well as other aspects and embodiments described elsewhere herein, the DLL4 antagonist is an antibody that competes for specific binding to the extracellular domain of human DLL4 with an antibody encoded by the plasmid deposited with ATCC having deposit no. PTA-8425. In some embodiments, the DLL4 antagonist is an antibody that competes for specific binding to human DLL4 with an antibody encoded by the plasmid deposited with ATCC having deposit no. PTA-8427. In some embodiments, the DLL4 antagonist is an antibody that competes for specific binding to human DLL4 with an antibody produced by the hybridoma deposited with ATCC having deposit no. PTA-8670. In some embodiments, the DLL4 antagonist is an antibody that competes for specific binding to the extracellular domain of human DLL4 with antibody 21M18, 21M18 H7L2 or 21M18 H9L2.

In certain embodiments of each of the aforementioned aspects, as well as other aspects and embodiments described elsewhere herein, the treatment methods further comprise administering at least one additional therapeutic agent appropriate for effecting combination therapy. In some embodiments, the additional therapeutic agent is a chemotherapeutic agent. In some embodiments, the chemotherapeutic agent is irinotecan, gemcitabine or 5-fluorouracil.

Pharmaceutical compositions comprising a DLL4 antagonist as described herein and a pharmaceutically acceptable vehicle are further provided, as are cell lines that produce the DLL4 antagonists. Methods of inhibiting tumor growth in a subject and/or treating cancer comprising administering to the subject an effective amount of a composition comprising DLL4 antagonists are also provided.

Where aspects or embodiments of the invention are described in terms of a Markush group or other grouping alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claims invention.

### BRIEF DESCRIPTION OF THE DRAWINGS

FIGS. 1A-1I. Inhibition of tumor growth in K-ras wild-type and mutant tumors with anti-EGFR antibody. C8 (FIGS. 1A and 1H), C40 (FIG. 1B), C4 (FIG. 1C), C6 (FIG. 1D), C9 (FIG. 1E), C12 (FIGS. 1F and 1I) and C22 (FIG. 1G) colon tumor cells were injected subcutaneously into NOD/SCID mice. Mice were treated with control antibody () or anti-EFGR antibody (▴) in FIGS. 1A-1G. Mice were treated with control antibody (), anti-EFGR antibody alone (▴), irinotecan alone (▾), or anti-EGFR antibody+irinotecan (∘) in FIGS. 1H-1I. Data is shown as tumor volume (mm³) over days post-treatment. Anti-EGFR antibody is cetuximab.

FIGS. 2A-2G. Inhibition of tumor growth in K-ras wild-type and mutant tumors with anti-DLL4 antibody, alone or in combination with irinotecan. C8 (FIG. 2A), C40 (FIG. 2B), C4 (FIG. 2C), C6 (FIG. 2D), C9 (FIG. 2E), C12 (FIG. 2F) and C22 (FIG. 2G) colon tumor cells were injected subcutaneously into NOD/SCID mice. Mice were treated with control antibody (), anti-DLL4 antibody alone (▪), irinotecan alone (▾), or anti-DLL4 antibody+irinotecan (♦). Data is shown as tumor volume (mm³) over days post-treatment. Anti-DLL4 antibody is a 1:1 mixture of 21M18 H7L2antibody (anti-human DLL4) and 21R30 antibody (anti-mouse DLL4).

FIG. 3. Cancer stem cell (CSC) frequency in C9 colon tumors following treatment with control antibody, anti-DLL4 antibody alone, irinotecan alone, or the combination of anti-DLL4 antibody and irinotecan, as determined by limiting dilution analysis. Anti-DLL4 antibody is a 1:1 mixture of 21M18 H7L2 antibody (anti-human DLL4) and 21R30 antibody (anti-mouse DLL4).

FIG. 4. Inhibition of tumor growth with anti-DLL4 antibody in a colon tumor recurrence xenograft model. C9 colon tumor cells were injected subcutaneously into NOD/SCID mice. Starting two days after injection, mice were treated with irinotecan alone (Δ) or anti-DLL4+irinotecan (▾). Treatments were discontinued on the indicated day and tumor growth was monitored for an additional period of time. Data is shown as tumor volume (mm³) over days post injection. Anti-DLL4 antibody is only 21M18 H7L2 antibody (anti-human DLL4).

FIG. 5. Inhibition of tumor growth with anti-DLL4 antibody in a pancreatic tumor xenograft model. PN8 pancreatic tumor cells were injected subcutaneously into NOD/SCID mice and allowed to grow for 28 days. Mice were treated with gemcitabine for 4 weeks after which gemcitabine treatments were stopped and antibody treatments initiated. Mice were treated with control antibody (), anti-mouse DLL4 antibody 21R30 (▾), anti-human DLL4 antibody 21M18 H7L2 (▴) or a combination of anti-mouse DLL4 21R30 and anti-human DLL4 antibodies 21M18 H7L2 (□). Data is shown as tumor volume (mm³) over days post-injection.

### DETAILED DESCRIPTION OF THE INVENTION

The present invention provides methods of inhibiting tumor growth, methods of treating cancer, and methods of reducing the frequency of cancer stem cells in a tumor. Particularly, the methods are directed to tumors or cancers that comprise a K-ras mutation. The methods provided herein comprise administering a DLL4 antagonist to a subject. In some embodiments, the DLL4 antagonist is an antibody that specifically binds the extracellular domain of human DLL4. Related polypeptides and polynucleotides, compositions comprising the DLL4 antagonists, and methods of making the DLL4 antagonists are also provided.

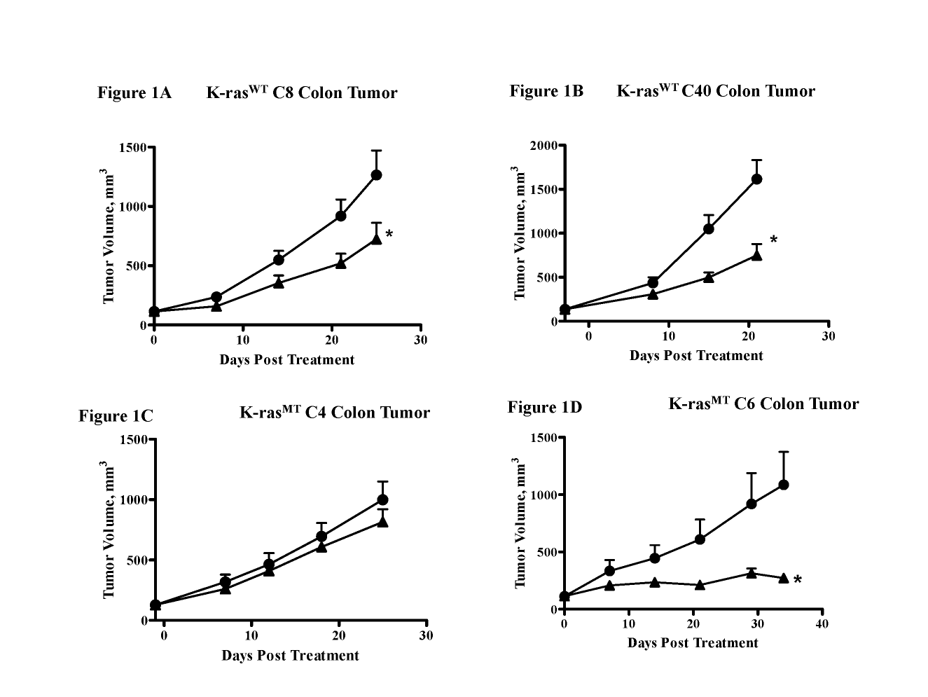

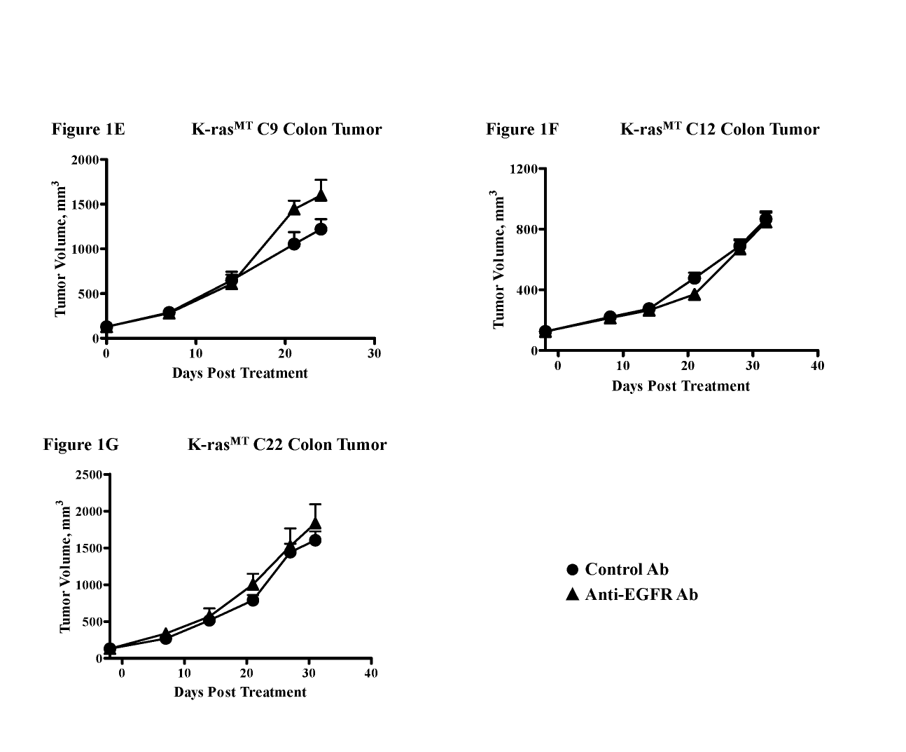

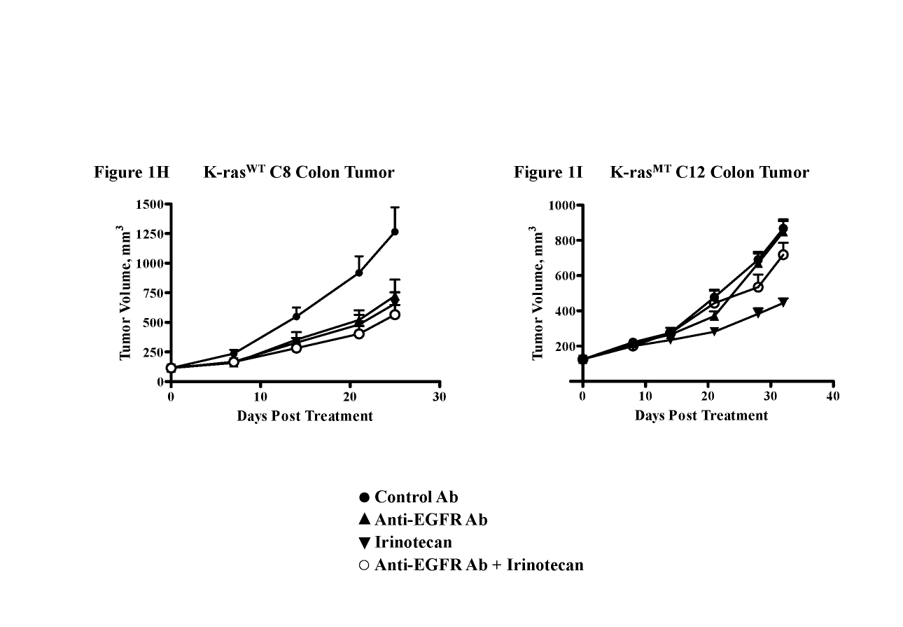

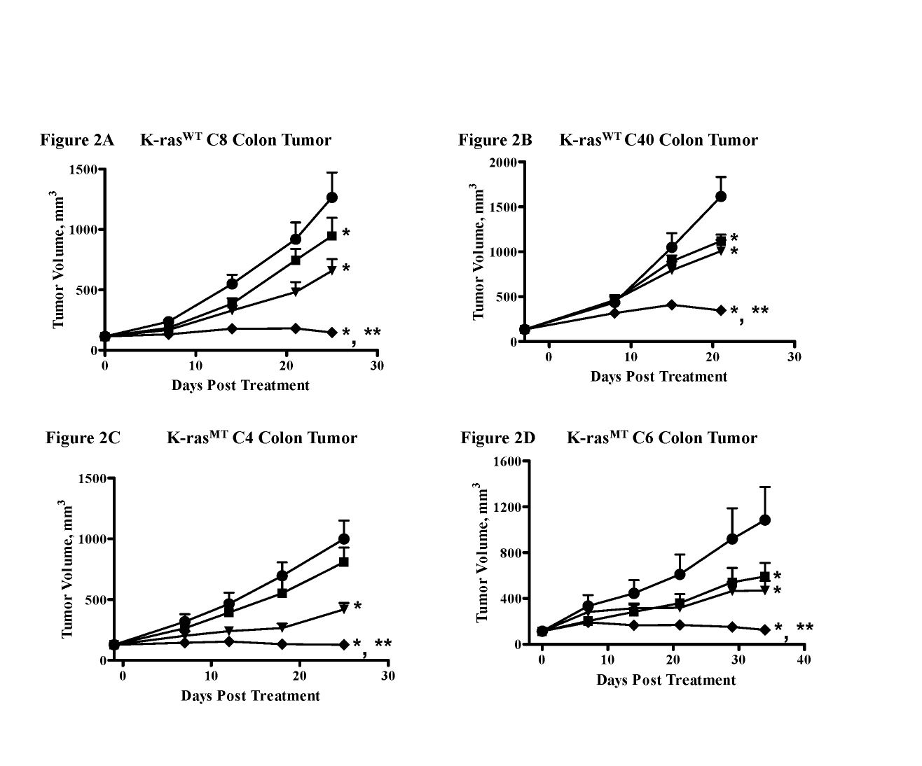

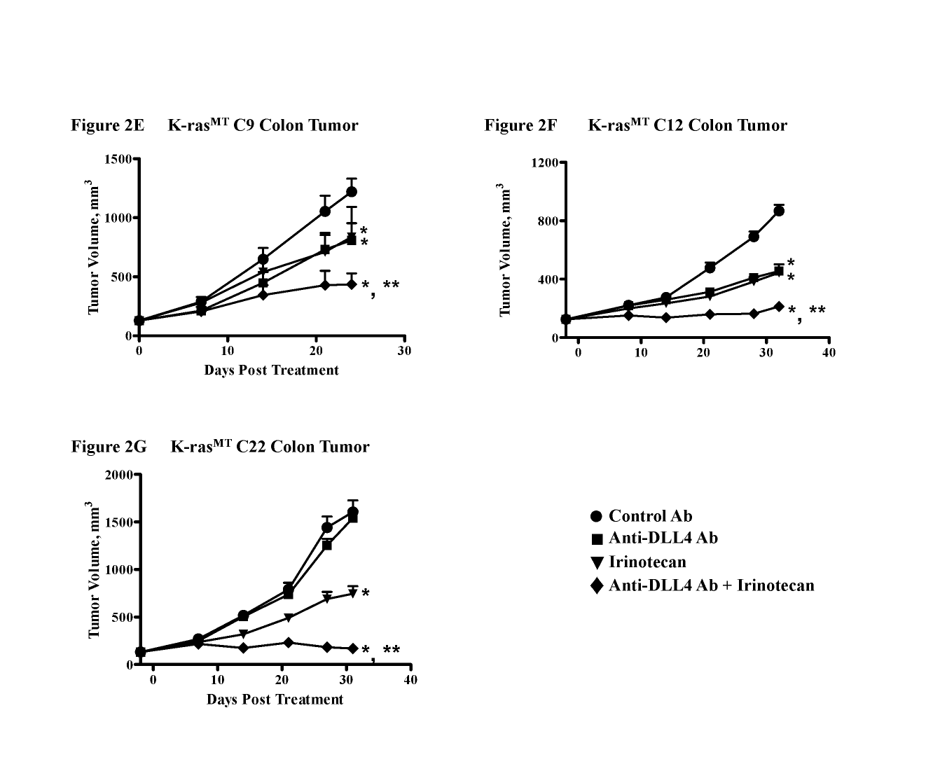

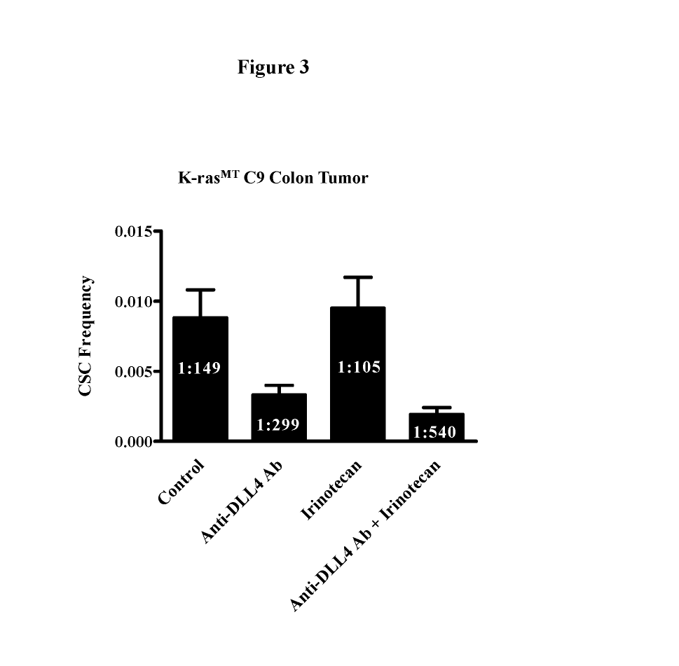

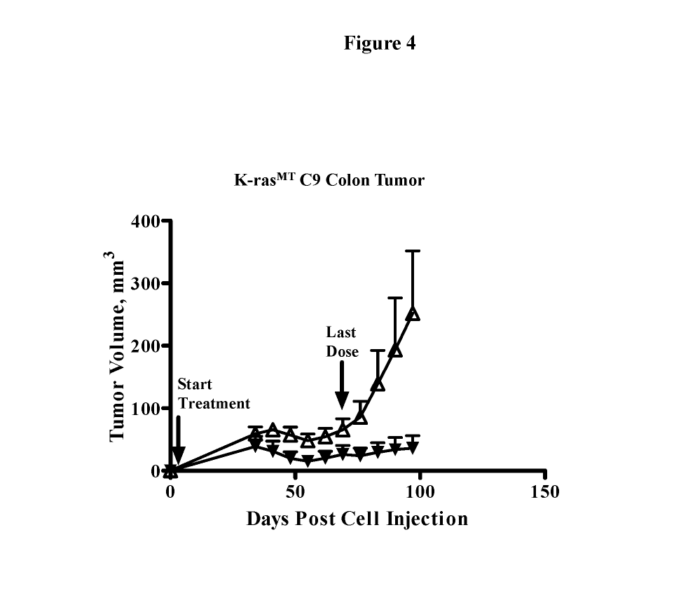

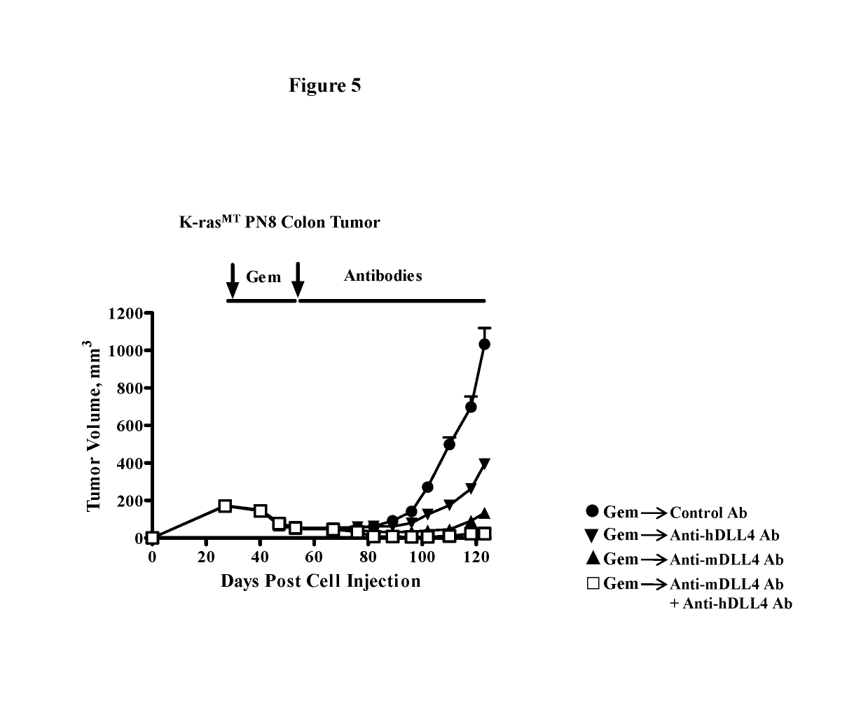

A number of colon tumors with wild-type K-ras or K-ras mutations were identified (Example 1). The colon tumors comprising K-ras mutations were shown to be non-responsive to anti-EGFR antibodies (Example 2 and FIGS. 1A-1I). Two of five of the colon tumors comprising K-ras mutations were responsive to anti-DLL4 antibodies alone, while all five were responsive to anti-DLL4 antibodies in combination with a chemotherapeutic agent (Example 2 and FIGS. 2A-2G). Anti-DLL4 antibodies, either alone or in combination with a chemotherapeutic agent, were also shown to reduce the frequency of cancer stem cells in a colon tumor comprising a K-ras mutation (Example 3 and FIG. 3). Treatment with anti-human DLL4 antibodies in combination with a chemotherapeutic agent was shown to inhibit growth of a K-ras mutant colon tumor even after discontinuation of the treatment (Example 4 and FIG. 4). In addition, anti-DLL4 antibodies were shown to inhibit growth of a K-ras mutant pancreatic tumor after initial treatment with a chemotherapeutic agent (Example 5 and FIG. 5).

### I. DEFINITIONS

To facilitate an understanding of the present invention, a number of terms and phrases are defined below.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site or antigen-binding site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen recognition site of an antibody, and any other modified immunoglobulin molecule comprising an antigen recognition site so long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy chain constant domains referred to as alpha, delta, epsilon, gamma, and mu, respectively. The different classes of immunoglobulins have different and well known subunit structures and three-dimensional configurations. Antibodies can be naked or conjugated to other molecules including, but not limited to, toxins and radioisotopes.

The term “antibody fragment” refers to a portion of an intact antibody and refers to the antigenic determining variable regions of an intact antibody. Examples of antibody fragments include, but are not limited to Fab, Fab′, F(ab′)2, and Fv fragments, linear antibodies, single chain antibodies, and multispecific antibodies formed from antibody fragments.

The term “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of the antibody. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-Lazikani et al., 1997, J. Molec. Biol. 273:927-948). In addition, combinations of these two approaches are sometimes used in the art to determine CDRs.

The term “monoclonal antibody” refers to a homogeneous antibody population involved in the highly specific recognition and binding of a single antigenic determinant, or epitope. This is in contrast to polyclonal antibodies that typically include a mixture of different antibodies directed against different antigenic determinants. The term “monoclonal antibody” encompasses both intact and full-length monoclonal antibodies as well as antibody fragments (such as Fab, Fab′, F(ab′)2, Fv fragments), single chain Fv (scFv) mutants, fusion proteins comprising an antibody portion, and any other modified immunoglobulin molecule comprising an antigen recognition site. Furthermore, “monoclonal antibody” refers to such antibodies made in any number of manners including, but not limited to, by hybridoma production, phage selection, recombinant expression, and transgenic animals.

The term “humanized antibody” refers to forms of non-human (e.g., murine) antibodies that are specific immunoglobulin chains, chimeric immunoglobulins, or fragments thereof that contain minimal non-human (e.g., murine) sequences.

The term “human antibody” means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammal (e.g., mouse, rat, rabbit, etc.) with the desired specificity, affinity, and/or capability while the constant regions are homologous to the sequences in antibodies derived from another species (usually human) to avoid eliciting an immune response in that species.

The terms “epitope” or “antigenic determinant” are used interchangeably herein and refer to that portion of an antigen capable of being recognized and specifically bound by a particular antibody. When the antigen is a polypeptide, epitopes can be formed both from contiguous amino acids (often referred to as “linear epitopes”) and noncontiguous amino acids juxtaposed by tertiary folding of a protein (often referred to as “conformation epitopes”). Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

The terms “specifically binds” or “specific binding” mean that a binding agent or an antibody reacts or associates more frequently, more rapidly, with greater duration, with greater affinity, or with some combination of the above to an epitope or protein than with alternative substances, including unrelated proteins. In certain embodiments, “specifically binds” means, for instance, that an antibody binds to a protein with a K_{D} of about 0.1 mM or less, but more usually less than about 1 μM. In certain embodiments, “specifically binds” means that an antibody binds to a protein at times with a K_{D} of at least about 0.1 μM or less, and at other times at least about 0.01 μM or less. Because of the sequence identity between homologous proteins in different species, specific binding can include an antibody that recognizes a particular protein such as DLL4 in more than one species (e.g., mouse DLL4 and human DLL4). It is understood that an antibody or binding moiety that specifically binds to a first target may or may not specifically bind to a second target. As such, “specific binding” does not necessarily require (although it can include) exclusive binding, i.e. binding to a single target. Thus, an antibody may, in certain embodiments, specifically bind to more than one target. In certain embodiments, the multiple targets may be bound by the same antigen-binding site on the antibody. For example, an antibody may, in certain instances, comprise two identical antigen-binding sites, each of which specifically binds the same epitope on two or more proteins. In certain alternative embodiments, an antibody may be bispecific and comprise at least two antigen-binding sites with differing specificities. By way of non-limiting example, a bispecific antibody may comprise one antigen-binding site that recognizes an epitope on a DLL4 protein, and further comprises a second, different antigen-binding site that recognizes a different epitope on a second protein, such as Notch. Generally, but not necessarily, reference to binding means specific binding.

The terms “polypeptide” or “peptide” or “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

The terms “polynucleotide” or “nucleic acid,” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”; substitution of one or more of the naturally occurring nucleotides with an analog; internucleotide modifications such as uncharged linkages (e.g., methyl phosphonates, phosphotriesters, phosphoamidates, cabamates, etc.) and charged linkages (e.g., phosphorothioates, phosphorodithioates, etc.); pendant moieties, such as proteins (e.g., nucleases, toxins, antibodies, signal peptides, poly-L-lysine, etc.); intercalators (e.g., acridine, psoralen, etc.); chelators (e.g., metals, radioactive metals, boron, oxidative metals, etc.); alkylators; modified linkages (e.g., alpha anomeric nucleic acids, etc.); as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha-anomeric sugars, epimeric sugars such as arabinose, xyloses or lyxoses, pyranose sugars, furanose sugars, heptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical.

“Conditions of high stringency” may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software are known in the art that may be used to obtain alignments of amino acid or nucleotide sequences. These include, but are not limited to, BLAST, ALIGN, Megalign, and BestFit. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 residues in length or any integral value therebetween. In some embodiments, identity exists over a longer region than 60-80 residues, such as at least about 90-100 residues, and in some embodiments the sequences are substantially identical over the full length of the sequences being compared, such as the coding region of a nucleotide sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Preferably, conservative substitutions in the sequences of the polypeptides and antibodies of the invention do not abrogate the binding of the polypeptide or antibody containing the amino acid sequence, to the antigen(s), i.e., the DLL4 protein to which the polypeptide or antibody binds. Methods of identifying nucleotide and amino acid conservative substitutions which do not eliminate antigen binding are well-known in the art.

The term “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, and DNA or RNA expression vectors encapsulated in liposomes.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure.

As used herein, “substantially pure” refers to material which is at least 50% pure (i.e., free from contaminants), more preferably at least 90% pure, more preferably at least 95% pure, more preferably at least 98% pure, more preferably at least 99% pure.

The terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers.

The terms “tumor” and “neoplasm” refer to any mass of tissue that results from excessive cell growth or proliferation, either benign (noncancerous) or malignant (cancerous) including pre-cancerous lesions.

The term “K-ras mutant” refers to a K-ras protein comprising at least one amino acid mutation as compared to wild-type K-ras (or to a nucleotide sequence encoding such a K-ras protein). K-ras mutants may include, but are not limited to, allelic variants, splice variants, substitution variants, deletion variants, and insertion variants. The term “K-ras mutation” refers to at least one amino acid mutation in the sequence of a K-ras protein as compared to the wild-type sequence (or to a nucleotide sequence encoding such a K-ras protein). The terms “K-ras mutant tumor” or “tumor comprising (or comprises) a K-ras mutation” are used interchangeably herein and refer to a population of tumor cells wherein a K-ras mutation can be detected, at either the protein or nucleotide level. The term “cancer comprising (or comprises) a K-ras mutation” as used herein refer to a population of cancer cells wherein a K-ras mutation can be detected, at either the protein or nucleotide level. K-ras mutations can be detected by techniques and methods known to one of skill in the art including, but not limited to, PCR-based assays (e.g., polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays), direct sequencing, primer extension reactions, electrophoresis, oligonucleotide ligation assays, hybridization assays, TaqMan assays, SNP genotyping assays, high resolution melting assays and microarray analyses.

The term “activating mutation” refers to a mutation that results in constitutive activation of a protein, for example, K-ras, and constitutive activation of a signaling pathway. In some embodiments, a K-ras protein comprising an activating mutation initiates constitutive activity of several pathways including, but not limited to, the MAP kinase cascade and the PI3 kinase cascade. In some embodiments, constitutive activity by the K-ras mutant and signaling pathways contributes significantly to several aspects of the malignant phenotype, including deregulation of cellular proliferation, impaired differentiation, reduced apoptosis and prolonged cell survival.

The terms “cancer stem cell” or “CSC” or “tumor stem cell” or “solid tumor stem cell” or “tumorigenic stem cell” are used interchangeably herein and refer to a population of cells from a solid tumor that: (1) have extensive proliferative capacity; 2) are capable of asymmetric cell division to generate one or more kinds of differentiated progeny with reduced proliferative or developmental potential; and (3) are capable of symmetric cell divisions for self-renewal or self-maintenance. These properties confer on the “cancer stem cells” the ability to form palpable tumors upon serial transplantation into an immunocompromised host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” or “tumor cell,” and grammatical equivalents refer to the total population of cells derived from a tumor or a pre-cancerous lesion, including both non-tumorigenic cells, which comprise the bulk of the tumor cell population, and tumorigenic stem cells (cancer stem cells). As used herein, the term “tumor cell” will be modified by the term “non-tumorigenic” when referring solely to those tumor cells lacking the capacity to renew and differentiate to distinguish those tumor cells from cancer stem cells.

The term “tumorigenic” refers to the functional features of a solid tumor stem cell including the properties of self-renewal (giving rise to additional tumorigenic cancer stem cells) and proliferation to generate all other tumor cells (giving rise to differentiated and thus non-tumorigenic tumor cells) that allow solid tumor stem cells to form a tumor. These properties of self-renewal and proliferation to generate all other tumor cells confer on cancer stem cells the ability to form palpable tumors upon serial transplantation into an immunocompromised host (e.g., a mouse) compared to non-tumorigenic tumor cells, which are unable to form tumors upon serial transplantation. It has been observed that non-tumorigenic tumor cells may form a tumor upon primary transplantation into an immunocompromised host after obtaining the tumor cells from a solid tumor, but those non-tumorigenic tumor cells do not give rise to a tumor upon serial transplantation.

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The phrase “pharmaceutically acceptable salt” refers to a salt of a compound that is pharmaceutically acceptable and that possesses the desired pharmacological activity of the parent compound.

The phrase “pharmaceutically acceptable excipient, carrier or adjuvant” refers to an excipient, carrier or adjuvant that can be administered to a subject, together with at least one antagonist or antibody of the present disclosure, and which does not destroy the pharmacological and/or biological activity thereof and is nontoxic when administered in doses sufficient to deliver a therapeutic amount of the antagonist.

The phrase “pharmaceutically acceptable vehicle” refers to a diluent, adjuvant, excipient, or carrier with which at least one antagonist or antibody of the present disclosure is administered.

The term “therapeutically effective amount” refers to an amount of an antibody, polypeptide, polynucleotide, small organic molecule, or other drug effective to “treat” a disease or disorder in a subject or mammal. In the case of cancer, the therapeutically effective amount of the drug (e.g., an antibody) can reduce the number of cancer cells; reduce the tumor size; inhibit and/or stop cancer cell infiltration into peripheral organs including, for example, the spread of cancer into soft tissue and bone; inhibit and/or stop tumor metastasis; inhibit and/or stop tumor growth; relieve to some extent one or more of the symptoms associated with the cancer; reduce morbidity and mortality; improve quality of life; decrease tumorigenicity, tumorgenic frequency, or tumorgenic capacity of a tumor; reduce the number or frequency of cancer stem cells in a tumor; differentiate tumorigenic cells to a non-tumorigenic state; or a combination of such effects. To the extent the drug prevents growth and/or kills existing cancer cells, it can be referred to as cytostatic and/or cytotoxic.

Terms such as “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent and/or slow the development of a targeted pathologic condition or disorder. Thus, those in need of treatment include those already with the disorder; those prone to have the disorder; and those in whom the disorder is to be prevented. In certain embodiments, a subject is successfully “treated” for cancer according to the methods of the present invention if the patient shows one or more of the following: a reduction in the number of, or complete absence of, cancer cells; a reduction in the tumor size; inhibition of, or an absence of, cancer cell infiltration into peripheral organs including, for example, the spread of cancer into soft tissue and bone; inhibition of, or an absence of, tumor metastasis; inhibition of, or an absence of, tumor growth; relief of one or more symptoms associated with the specific cancer; reduced morbidity and mortality; improvement in quality of life; reduction in tumorigenicity, tumorgenic frequency, or tumorgenic capacity of a tumor; reduction in the number or frequency of cancer stem cells in a tumor; differentiation of tumorigenic cells to a non-tumorigenic state; or some combination of effects.

The phrase “substantially non-responsive” as used herein refers to a tumor or a cancer that shows stable growth or increased growth after administration of a therapeutic agent. The phrase may refer to a patient that shows stable disease or progressive disease after administration of a therapeutic agent. The phrase may be used when referring to tumors or cancers that are resistant to treatment with a therapeutic agent. The phrase “substantially non-responsive to an EGFR inhibitor” as used herein refers to a tumor or a cancer that shows stable growth or increased growth after administration of an EGFR inhibitor. In some embodiments, an EGFR inhibitor is administered to a patient in need of treatment, and “substantially non-responsive” to the EGFR inhibitor includes: no reduction in the number of, or continued growth of, cancer cells; no reduction in the tumor size; an increase in tumor size; no inhibition of, or a continuation of, cancer cell infiltration into peripheral organs including, for example, the spread of cancer into soft tissue and bone; no inhibition of, or a continuation of, tumor metastasis; no inhibition of, or a continuation of, tumor growth; no or little relief of one or more symptoms associated with the specific cancer; no or little reduction in tumorigenicity, tumorgenic frequency, or tumorgenic capacity of a tumor; no or little reduction in the number or frequency of cancer stem cells in a tumor; or some combination of effects.

As used in the present disclosure and claims, the singular forms “a” “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided.

The term “and/or” as used in a phrase such as “A and/or B” herein is intended to include both “A and B,” “A or B,” “A” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. DLL4 ANTAGONISTS

The present invention provides DLL4 antagonists for use in methods of inhibiting growth of a tumor, wherein the tumor comprises a K-ras mutation and/or wherein the tumor is substantially non-responsive to at least one epithelial growth factor receptor (EGFR) inhibitor. The invention further provides DLL4 antagonists for use in methods of treating cancer, wherein the cancer comprises a K-ras mutation and/or wherein the cancer is substantially non-responsive to at least one epithelial growth factor receptor (EGFR) inhibitor.

In certain embodiments, the DLL4 antagonist specifically binds the extracellular domain of human DLL4. In some embodiments, the DLL4 antagonist is an antibody. In some embodiments, the DLL4 antagonist or antibody specifically binds an epitope comprising amino acids within the N-terminal region of human DLL4 (SEQ ID NO:16). In some embodiments, the DLL4 antagonist or antibody specifically binds an epitope formed by a combination of the N-terminal region of human DLL4 (SEQ ID NO:16) and the DSL region of human DLL4 DSL region (SEQ ID NO:17).

In certain embodiments, the DLL4 antagonist (e.g., an antibody) binds to DLL4 with a dissociation constant (K_{D}) of about 1 μM or less, about 100 nM or less, about 40 nM or less, about 20 nM or less, about 10 nM or less or about 1 nM or less. In certain embodiments, the DLL4 antagonist or antibody binds to human DLL4 with a K_{D} of about 40 nM or less, about 20 nM or less, about 10 nM, or less or about 1 nM or less. In some embodiments, the dissociation constant of the antagonist or antibody to DLL4 is the dissociation constant determined using a DLL4 fusion protein comprising a DLL4 extracellular domain (e.g., a DLL4 ECD-Fc fusion protein) immobilized on a Biacore chip.

In certain embodiments, the DLL4 antagonist (e.g., an antibody) binds to DLL4 with a half maximal effective concentration (EC₅₀) of about 1 μM or less, about 100 nM or less, about 40 nM or less, about 20 nM or less, about 10 nM or less, or about 1 nM or less. In certain embodiments, the DLL4 antagonist or antibody binds to human DLL4 with an EC₅₀ of about 40 nM or less, about 20 nM or less, about 10 nM or less, or about 1 nM or less.

In certain embodiments, the DLL4 antagonist is a polypeptide. In certain embodiments, the DLL4 antagonist or polypeptide is an antibody. In certain embodiments, the antibody is an IgG antibody. In some embodiments, the antibody is an IgG1 antibody. In some embodiments, the antibody is an IgG2 antibody. In certain embodiments, the antibody is a monoclonal antibody. In certain embodiments, the antibody is a humanized antibody. In certain embodiments, the antibody is a human antibody. In certain embodiments, the antibody is an antibody fragment.

The DLL4 antagonists (e.g., antibodies) of the present invention can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blot analysis, radioimmunoassay, ELISA, “sandwich” immunoassay, immunoprecipitation assay, precipitation reaction, gel diffusion precipitin reaction, immunodiffusion assay, agglutination assay, complement-fixation assay, immunoradiometric assay, fluorescent immunoassay, and protein A immunoassay. Such assays are routine and well known in the art (see, e.g., Ausubel et al., eds, 1994, Current Protocols in Molecular Biology, Vol. 1, John Wiley & Sons, Inc., New York).

In some embodiments, the specific binding of a DLL4 antagonist (e.g., an antibody) to human DLL4 may be determined using ELISA. An ELISA assay comprises preparing DLL4 antigen, coating wells of a 96 well microtiter plate with antigen, adding to the wells the DLL4 antagonist or antibody conjugated to a detectable compound such as an enzymatic substrate (e.g., horseradish peroxidase or alkaline phosphatase), incubating for a period of time and detecting the presence of the binding agent or antibody. In some embodiments, the DLL4 antagonist or antibody is not conjugated to a detectable compound, but instead a second conjugated antibody that recognizes the DLL4 antagonist or antibody is added to the well. In some embodiments, instead of coating the well with DLL4 antigen, the DLL4 antagonist or antibody can be coated to the well, antigen is added to the coated well and then a second antibody conjugated to a detectable compound is added. One of skill in the art would be knowledgeable as to the parameters that can be modified and/or optimized to increase the signal detected, as well as other variations of ELISAs that can be used (see e.g., Ausubel et al., eds, 1994, Current Protocols in Molecular Biology, Vol. 1, John Wiley & Sons, Inc., New York at 11.2.1).

The binding affinity of an antagonist or antibody to DLL4 and the on-off rate of an antibody-antigen interaction can be determined by competitive binding assays. In some embodiments, a competitive binding assay is a radioimmunoassay comprising the incubation of labeled antigen (e.g., ³H or ¹²⁵I), or fragment or variant thereof, with the antibody of interest in the presence of increasing amounts of unlabeled antigen followed by the detection of the antibody bound to the labeled antigen. The affinity of the antibody for the antigen and the on-off rates can be determined from the data by Scatchard plot analysis. In some embodiments, Biacore kinetic analysis is used to determine the binding affinities and on-off rates of antagonists or antibodies that bind DLL4. Biacore kinetic analysis comprises analyzing the binding and dissociation of antibodies from antigens (e.g., DLL4 proteins) that have been immobilized on the surface of a Biacore chip. In some embodiments, Biacore kinetic analyses can be used to study binding of different antibodies in qualitative epitope competition binding assays.

In some embodiments, the DLL4 antagonists are polyclonal antibodies. Polyclonal antibodies can be prepared by any known method. Polyclonal antibodies are prepared by immunizing an animal (e.g., a rabbit, rat, mouse, goat, donkey, etc.) by multiple subcutaneous or intraperitoneal injections of the relevant antigen (e.g., a purified peptide fragment, full-length recombinant protein, fusion protein, etc.). The antigen can be optionally conjugated to a carrier protein such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from blood, ascites and the like, of the immunized animal. Polyclonal antibodies can be purified from serum or ascites according to standard methods in the art including, but not limited to, affinity chromatography, ion-exchange chromatography, gel electrophoresis, and dialysis.

In some embodiments, the DLL4 antagonists are monoclonal antibodies. Monoclonal antibodies can be prepared using hybridoma methods known to one of skill in the art (see e.g., Kohler and Milstein, 1975, Nature 256:495). Using the hybridoma method, a mouse, hamster, or other appropriate host animal, is immunized as described above to elicit from lymphocytes the production of antibodies that will specifically bind to the immunizing antigen. In some embodiments, lymphocytes can be immunized in vitro. In some embodiments, the immunizing antigen (e.g., DLL4) can be a human protein or a portion thereof. In some embodiments, the immunizing antigen (e.g., DLL4) can be a mouse protein or a portion thereof. In some embodiments, the immunizing antigen can be an extracellular domain of human DLL4. In some embodiments, the immunizing antigen can be an extracellular domain of mouse DLL4. In some embodiments, a mouse is immunized with a human antigen. In some embodiments, a mouse is immunized with a mouse antigen.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed against a chosen antigen may be identified by a variety of techniques including, but not limited to, immunoprecipitation, immunoblotting, and in vitro binding assays (e.g., flow cytometry, enzyme-linked immunosorbent assay (ELISA), or radioimmunoassay (RIA)). The hybridomas can be propagated either in in vitro culture using standard methods (Goding, Monoclonal Antibodies: Principles and Practice, Academic Press, 1986) or in in vivo as ascites in an animal. The monoclonal antibodies can be purified from the culture medium or ascites fluid according to standard methods in the art including, but not limited to, affinity chromatography, ion-exchange chromatography, gel electrophoresis, and dialysis.

Alternatively, monoclonal antibodies can be made using recombinant DNA techniques as known to one skilled in the art (see e.g., U.S. Pat. No. 4,816,567). The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional techniques. The isolated polynucleotides encoding the heavy and light chains are cloned into suitable expression vectors which produce the monoclonal antibodies when transfected into host cells such as E. coli, simian COS cells, Chinese hamster ovary (CHO) cells, or myeloma cells that do not otherwise produce immunoglobulin protein. Recombinant monoclonal antibodies, or fragments thereof, can also be isolated from phage display libraries expressing CDRs of the desired species (see e.g., McCafferty et al., 1990, Nature, 348:552-554; Clackson et al., 1991, Nature, 352:624-628; and Marks et al., 1991, J. Mol. Biol., 222:581-597).

The polynucleotide(s) encoding a monoclonal antibody can be further modified using recombinant DNA technology to generate alternative antibodies. In some embodiments, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, and/or other biological characteristics of a monoclonal antibody. In some embodiments, site-directed mutagenesis of the CDRs can be used to optimize specificity, affinity, and/or other biological characteristics of a monoclonal antibody.

In some embodiments, the DDL4 antagonist is a humanized antibody. Typically, humanized antibodies are human immunoglobulins in which residues from the complementary determining regions (CDRs) are replaced by residues from CDRs of a non-human species (e.g., mouse, rat, rabbit, hamster) that have the desired specificity, affinity, and/or capability by methods known to one skilled in the art. In some embodiments, the Fv framework region (FR) residues of a human immunoglobulin are replaced with the corresponding framework region residues from a non-human immunoglobulin that has the desired specificity, affinity, and/or capability. In some embodiments, the humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all, or substantially all, of the CDRs that correspond to the non-human immunoglobulin whereas all, or substantially all, of the framework regions are those of a human immunoglobulin consensus sequence. In some embodiments, the humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject. One skilled in the art would be able to obtain a functional humanized antibody with reduced immunogenicity following known techniques (see for example U.S. Pat. Nos. 5,225,539; 5,585,089; 5,693,761; and 5,693,762).

In some embodiments, the invention provides an antibody that specifically binds the extracellular domain of human DLL4, wherein the antibody comprises one, two, three, four, five and/or six of the CDRs of antibodies 21M18, 21M18 H9L2 and/or 21M18 H7L2. These antibodies have been described in U.S. Patent Application No. 2008/0187532. Antibodies 21M18 H7L2 and 21M18 H9L2 are humanized forms of the murine 21M18 antibody.

In certain embodiments, the invention provides a DLL4 antagonist, wherein the antagonist is a DLL4 antibody that specifically binds the extracellular domain of human DLL4, and wherein the antibody comprises: a heavy chain CDR1 comprising TAYYIH (SEQ ID NO:1), a heavy chain CDR2 comprising YISCYNGATNYNQKFKG (SEQ ID NO:2), YISSYNGATNYNQKFKG (SEQ ID NO:3), or YISVYNGATNYNQKFKG (SEQ ID NO:4), and a heavy chain CDR3 comprising RDYDYDVGMDY (SEQ ID NO:5). In some embodiments, the antibody further comprises a light chain CDR1 comprising RASESVDNYGISFMK (SEQ ID NO:7), a light chain CDR2 comprising AASNQGS (SEQ ID NO:8), and a light chain CDR3 comprising QQSKEVPWTFGG (SEQ ID NO:9). In some embodiments, the antibody comprises a light chain CDR1 comprising RASESVDNYGISFMK (SEQ ID NO:7), a light chain CDR2 comprising AASNQGS (SEQ ID NO:8), and a light chain CDR3 comprising QQSKEVPWTFGG (SEQ ID NO:9).

In certain embodiments, the invention provides an antibody that specifically binds the extracellular domain of human DLL4, wherein the antibody comprises a heavy chain variable region having at least about 80% sequence identity to SEQ ID NO:6, SEQ ID NO:12, or SEQ ID NO:13, and/or a light chain variable region having at least 80% sequence identity to SEQ ID NO:10. In certain embodiments, the antibody comprises a heavy chain variable region having at least about 85%, at least about 90%, at least about 95%, at least about 97%, or at least about 99% sequence identity to SEQ ID NO:6, SEQ ID NO:12 or SEQ ID NO:13. In certain embodiments, the antibody comprises a light chain variable region having at least about 85%, at least about 90%, at least about 95%, at least about 97%, or at least about 99% sequence identity to SEQ ID NO:10. In certain embodiments, the antibody comprises a heavy chain variable region having at least about 95% sequence identity to SEQ ID NO:6, SEQ ID NO:12 or SEQ ID NO:13, and/or a light chain variable region having at least about 95% sequence identity to SEQ ID NO:10. In certain embodiments, the antibody comprises a heavy chain variable region comprising SEQ ID NO:6, SEQ ID NO:12, or SEQ ID NO:13, and/or a light chain variable region comprising SEQ ID NO:10. In certain embodiments, the antibody comprises a heavy chain variable region comprising SEQ ID NO:6 and a light chain variable region comprising SEQ ID NO:10. In certain embodiments, the antibody comprises a heavy chain variable region comprising SEQ ID NO:12 and a light chain variable region comprising SEQ ID NO:10. In certain embodiments, the antibody comprises a heavy chain variable region comprising SEQ ID NO:13 and a light chain variable region comprising SEQ ID NO:10.

In certain embodiments, the DLL4 antagonist (e.g., an antibody) binds to the same epitope that an antibody comprising the heavy chain variable region comprising SEQ ID NO:6, and/or a light chain variable region comprising SEQ ID NO:10 binds. In certain embodiments, the DLL4 antagonist (e.g., an antibody) binds to the same epitope that an antibody comprising the heavy chain variable region comprising SEQ ID NO:12, and/or a light chain variable region comprising SEQ ID NO:10 binds. In certain embodiments, the DLL4 antagonist (e.g., an antibody) binds to the same epitope that an antibody comprising the heavy chain variable region comprising SEQ ID NO:13, and/or a light chain variable region comprising SEQ ID NO:10 binds. In some embodiments, the DLL4 antagonist or antibody binds to the same epitope as antibody 21M18. In some embodiments, the DLL4 antagonist or antibody binds to the same epitope as antibody 21M18 H7L2. In some embodiments, the DLL4 antagonist or antibody binds to the same epitope as antibody 21M18 H9L2.

In certain embodiments, the DLL4 antagonist (e.g., an antibody) competes for specific binding to an extracellular domain of human DLL4 with an antibody, wherein the antibody comprises a heavy chain variable region comprising SEQ ID NO:6, and/or a light chain variable region comprising SEQ ID NO:10. In certain embodiments, the DLL4 antagonist competes for specific binding to an extracellular domain of human DLL4 with an antibody, wherein the antibody comprises a heavy chain variable region comprising SEQ ID NO:12, and/or a light chain variable region comprising SEQ ID NO:10. In certain embodiments, the DLL4 antagonist competes for specific binding to an extracellular domain of human DLL4 with an antibody, wherein the antibody comprises a heavy chain variable region comprising SEQ ID NO:13, and/or a light chain variable region comprising SEQ ID NO:10. In some embodiments, the DLL4 antagonist competes for specific binding to an extracellular domain of human DLL4 with an antibody encoded by the plasmid deposited with ATCC having deposit no. PTA-8425. In some embodiments, the DLL4 antagonist or antibody competes for specific binding to an extracellular domain of human DLL4 with an antibody encoded by the plasmid deposited with ATCC having deposit no. PTA-8427. In some embodiments, the DLL4 antagonist or antibody competes for specific binding to an extracellular domain of human DLL4 with an antibody produced by the hybridoma deposited with ATCC having deposit no. PTA-8670. In some embodiments, the DLL4 antagonist or antibody competes for specific binding to an extracellular domain of human DLL4 in a competitive binding assay.

In certain embodiments, the DDL4 antagonist is a human antibody. Human antibodies can be directly prepared using various techniques known in the art. Immortalized human B lymphocytes, immunized in vitro or isolated from an immunized individual, that produce an antibody directed against a target antigen can be generated. Alternatively, a human antibody can be selected from a phage library, where that phage library expresses human antibodies (see e.g., Vaughan et al., 1996, Nat. Biotech., 14:309-314; Sheets et al., 1998, Proc. Nat'l. Acad. Sci., 95:6157-6162; Hoogenboom and Winter, 1991, J. Mol. Biol., 227:381; and Marks et al., 1991, J. Mol. Biol., 222:581). Techniques for the generation and use of antibody phage libraries are also described in U.S. Pat. Nos. 5,969,108; 6,172,197; 5,885,793; 6,521,404; 6,544,731; 6,555,313; 6,582,915; 6,593,081; 6,300,064; 6,653,068; 6,706,484; and 7,264,963; and Rothe et al., 2008, J. Mol. Bio., 376:1182-1200. Affinity maturation strategies, such as chain shuffling (Marks et al., 1992, Bio/Technology, 10:779-783), are known in the art and may be employed to generate high affinity human antibodies.

Human antibodies can also be made in transgenic mice containing human immunoglobulin loci that are capable, upon immunization, of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In certain embodiments, the DDL4 antagonist is a bispecific antibody. Bispecific antibodies are capable of specifically recognizing and binding to at least two different epitopes. The different epitopes can either be within the same molecule or on different molecules. In some embodiments, the antibodies can specifically recognize and bind a first antigen target, (e.g., DLL4) as well as a second antigen target, such as an effector molecule on a leukocyte (e.g., CD2, CD3, CD28, or B7) or a Fc receptor (e.g., CD64, CD32, or CD16) so as to focus cellular defense mechanisms to the cell expressing the first antigen target. In some embodiments, the antibodies can be used to direct cytotoxic agents to cells which express a particular target antigen, such as DLL4. These antibodies possess an antigen-binding arm and an arm which binds a cytotoxic agent or a radionuclide chelator, such as EOTUBE, DPTA, DOTA, or TETA. In certain embodiments, the bispecific antibody specifically binds DLL4, as well as either VEGF, a second Notch ligand (e.g., Jagged1 or Jagged2), or at least one Notch receptor selected from the group consisting of Notch1, Notch2, Notch3, and Notch4.

Techniques for making bispecific antibodies are known by those skilled in the art, see for example, Millstein et al., 1983, Nature, 305:537-539; Brennan et al., 1985, Science, 229:81; Suresh et al, 1986, Methods in Enzymol., 121:120; Traunecker et al., 1991, EMBO J., 10:3655-3659; Shalaby et al., 1992, J. Exp. Med., 175:217-225; Kostelny et al., 1992, J. Immunol., 148:1547-1553; Gruber et al., 1994, J. Immunol., 152:5368; and U.S. Pat. No. 5,731,168). Bispecific antibodies can be intact antibodies or antibody fragments. Antibodies with more than two valencies are also contemplated. For example, trispecific antibodies can be prepared (Tutt et al., 1991, J. Immunol., 147:60). Thus, in certain embodiments the antibodies to DLL4 are multispecific.

In certain embodiments, the DDL4 antagonists (e.g., antibodies or other polypeptides) described herein may be monospecific. For example, in certain embodiments, each of the one or more antigen-binding sites that an antibody contains is capable of binding (or binds) a homologous epitope on DLL4.

In certain embodiments, the DDL4 antagonist is an antibody fragment. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced recombinantly. In some embodiments, antibody fragments include Fv or single chain Fv (scFv) fragments. Fab, Fv, and scFv antibody fragments can be expressed in, and secreted from, E. coli or other host cells, allowing for the production of large amounts of these fragments. In some embodiments, antibody fragments are isolated from antibody phage libraries as discussed herein. For example, methods can be used for the construction of Fab expression libraries (Huse et al., 1989, Science, 246:1275-1281) to allow rapid and effective identification of monoclonal Fab fragments with the desired specificity for DLL4, or derivatives, fragments, analogs or homologs thereof. In some embodiments, antibody fragments are linear antibody fragments as described in U.S. Pat. No. 5,641,870. In certain embodiments, antibody fragments are monospecific or bispecific. In certain embodiments, the DDL4 antagonist is a scFv. Various techniques can be used for the production of single-chain antibodies specific to DLL4 (see, e.g., U.S. Pat. No. 4,946,778).

It can further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

For the purposes of the present invention, it should be appreciated that modified antibodies, or fragments thereof, can comprise any type of variable region that provides for the association of the antibody with DLL4. In this regard, the variable region may be derived from any type of mammal that can be induced to mount a humoral response and generate immunoglobulins against a desired antigen (e.g., DLL4). As such, the variable region of the modified antibodies can be, for example, of human, murine, non-human primate (e.g., cynomolgus monkeys, macaques, etc.) or lapine origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence modification. Although the CDRs may be derived from an antibody of the same class or even subclass as the antibody from which the framework regions are derived, it is envisaged that the CDRs will be derived from an antibody of a different class and preferably from an antibody from a different species. It may not be necessary to replace all of the CDRs with all of the CDRs from the donor variable region to transfer the antigen binding capacity of one variable domain to another. Rather, it may only be necessary to transfer those residues that are necessary to maintain the activity of the antigen binding site.

Alterations to the variable region notwithstanding, those skilled in the art will appreciate that the modified antibodies of this invention will comprise antibodies (e.g., full-length antibodies or antigen-binding fragments thereof) in which at least a fraction of one or more of the constant region domains has been deleted or otherwise altered so as to provide desired biochemical characteristics, such as increased tumor localization, increased tumor penetration, reduced serum half-life or increased serum half-life when compared with an antibody of approximately the same immunogenicity comprising a native or unaltered constant region. In some embodiments, the constant region of the modified antibodies comprises a human constant region. Modifications to the constant region include additions, deletions or substitutions of one or more amino acids in one or more domains. The modified antibodies disclosed herein may comprise alterations or modifications to one or more of the three heavy chain constant domains (CHL CH2 or CH3) and/or to the light chain constant domain (CL). In some embodiments, one or more domains are partially or entirely deleted from the constant regions of the modified antibodies. In some embodiments, the entire CH2 domain has been removed (ΔCH2 constructs). In some embodiments, the omitted constant region domain is replaced by a short amino acid spacer (e.g., 10 aa residues) that provides some of the molecular flexibility typically imparted by the absent constant region.

In certain embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers can, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding and thereby increase tumor localization and/or tumor penetration. Similarly, it may be desirable to simply delete the part of one or more constant region domains that control a specific effector function (e.g., complement C1q binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody can bind to a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (ADCC), release of inflammatory mediators, placental transfer and control of immunoglobulin production.

In certain embodiments, the DLL4 antibodies provide for altered effector functions that, in turn, affect the biological profile of the administered antibody. For example, in some embodiments, the deletion or inactivation (through point mutations or other means) of a constant region domain may reduce Fc receptor binding of the circulating modified antibody (e.g., DLL4 antibody) thereby increasing tumor localization and/or penetration. In other embodiments, the constant region modifications increase or reduce the serum half-life of the antibody. In some embodiments, the constant region is modified to eliminate disulfide linkages or oligosaccharide moieties allowing for enhanced tumor localization and/or penetration.

In certain embodiments, a DLL4 antibody does not have one or more effector functions. In some embodiments, the antibody has no antibody-dependent cellular cytoxicity (ADCC) activity and/or no complement-dependent cytoxicity (CDC) activity. In certain embodiments, the antibody does not bind to an Fc receptor and/or complement factors. In certain embodiments, the antibody has no effector function.

The present invention further embraces variants and equivalents which are substantially homologous to the chimeric, humanized and human antibodies, or antibody fragments thereof, set forth herein. These can contain, for example, conservative substitution mutations, i.e. the substitution of one or more amino acids by similar amino acids.

Thus, the present invention provides methods for generating an antibody that binds the extracellular domain of human DLL4. In some embodiments, the method for generating an antibody that binds DLL4 comprises using hybridoma techniques. In some embodiments, the method comprises using an extracellular domain of mouse DLL4 or human DLL4 as an immunizing antigen. In some embodiments, the method of generating an antibody that binds DLL4 comprises screening a human phage library. The present invention further provides methods of identifying an antibody that binds to DLL4. In some embodiments, the antibody is identified by screening for binding to DLL4 with flow cytometry (FACS). In some embodiments, the antibody is screened for binding to human DLL4. In some embodiments, the antibody is screened for binding to mouse DLL4. In some embodiments, the antibody is identified by screening for inhibition or blocking of DLL4-induced Notch activation. In some embodiments, the DLL4 is human DLL4. In some embodiments, the Notch is human Notch1, Notch2, Notch3 or Notch4.

In certain embodiments, the antibodies as described herein are isolated. In certain embodiments, the antibodies as described herein are substantially pure.

Certain anti-DLL4 antibodies have been described, for example, in U.S. Patent Application Pub. No. 2008/0187532, incorporated by reference herein in its entirety. Certain additional anti-DLL4 antibodies are described in, e.g., International Patent Publication Nos. WO 2008/091222 and WO 2008/0793326, and U.S. Patent Application Publication Nos. 2008/0014196; 2008/0175847; 2008/0181899; and 2008/0107648, each of which is incorporated by reference herein in its entirety.

In some embodiments of the present invention, the DLL4 antagonists are polypeptides. The polypeptides can be recombinant polypeptides, natural polypeptides, or synthetic polypeptides that bind DLL4. In some embodiments, the polypeptides comprise an antibody or fragment thereof that binds DLL4. It will be recognized by those in the art that some amino acid sequences of a polypeptide can be varied without significant effect on the structure or function of the protein. Thus, the polypeptides further include variations of the polypeptides which show substantial binding activity against DLL4 protein. In some embodiments, amino acid sequence variations of polypeptides include deletions, insertions, inversions, repeats, and/or type substitutions.

The polypeptides and variants thereof, can be further modified to contain additional chemical moieties not normally part of the polypeptide. The derivatized moieties can improve the solubility, the biological half-life or absorption of the polypeptide. The moieties can also reduce or eliminate any undesirable side effects of the polypeptides and variants. An overview for such chemical moieties can be found in Remington: The Science and Practice of Pharmacy, 21st Edition, University of the Sciences in Philadelphia, 2005.

The isolated polypeptides described herein can be produced by any suitable method known in the art. Such methods range from direct protein synthesis methods to constructing a DNA sequence encoding isolated polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional variants thereof.

In some embodiments, a DNA sequence encoding a polypeptide of interest may be constructed by chemical synthesis using an oligonucleotide synthesizer. Oligonucleotides can be designed based on the amino acid sequence of the desired polypeptide and by selecting those codons that are favored in the host cell in which the recombinant polypeptide of interest will be produced. Standard methods can be applied to synthesize a polynucleotide sequence encoding a polypeptide of interest. For example, a complete amino acid sequence can be used to construct a back-translated gene. Further, a DNA oligomer containing a nucleotide sequence coding for the particular polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis or another method), the polynucleotide sequences encoding a particular polypeptide of interest can be inserted into an expression vector and operatively linked to an expression control sequence appropriate for expression of the polypeptide in a desired host. Proper assembly can be confirmed by nucleotide sequencing, restriction mapping, and/or expression of a biologically active polypeptide in a suitable host. As is well known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding DLL4 antagonists such as polypeptides or antibodies, or fragments thereof. For example, recombinant expression vectors can be replicable DNA constructs which have synthetic or cDNA-derived DNA fragments encoding a polypeptide chain of an anti-DLL4 antibody, or fragment thereof, operatively linked to suitable transcriptional or translational regulatory elements derived from mammalian, microbial, viral or insect genes. A transcriptional unit generally comprises an assembly of (1) a regulatory element or elements having a role in gene expression, for example, transcriptional promoters and/or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of an expression vector and control elements depends upon the choice of host. A wide variety of expression host/vector combinations can be employed. Useful expression vectors for eukaryotic hosts include, for example, vectors comprising expression control sequences from SV40, bovine papilloma virus, adenovirus and cytomegalovirus. Useful expression vectors for bacterial hosts include known bacterial plasmids, such as plasmids from E. coli, including pCR1, pBR322, pMB9 and their derivatives and wider host range plasmids, such as M13 and other filamentous single-stranded DNA phages.

Suitable host cells for expression of a DLL4 antagonist polypeptide or antibody (or a DLL4 protein to use as an antigen) include prokaryotes, yeast, insect or higher eukaryotic cells under the control of appropriate promoters. Prokaryotes include gram-negative or gram-positive organisms, for example, E. coli or Bacilli. Higher eukaryotic cells include established cell lines of mammalian origin as described below. Cell-free translation systems can also be employed.

Various mammalian or insect cell culture systems are used to express recombinant protein. Expression of recombinant proteins in mammalian cells may be preferred because such proteins are generally correctly folded, appropriately modified and completely functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived) and BHK (hamster kidney fibroblast-derived) cell lines. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, 1988, Bio/Technology, 6:47.

The proteins produced by a transformed host can be purified according to any suitable method. Such methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence and glutathione-S-transferase can be attached to the protein to allow easy purification by passage over an appropriate affinity column. Isolated proteins can also be physically characterized using such techniques as proteolysis, high performance liquid chromatography (HPLC), nuclear magnetic resonance and x-ray crystallography.

For example, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin can be employed, for example, a matrix or substrate having pendant diethylaminoethyl (DEAE) groups. The matrices can be acrylamide, agarose, dextran, cellulose or other types commonly employed in protein purification. In some embodiments, a cation exchange step can be employed. Suitable cation exchangers include various insoluble matrices comprising sulfopropyl or carboxymethyl groups. In some embodiments, a hydroxyapatite (CHT) media can be employed, including but not limited to, ceramic hydroxyapatite. In some embodiments, one or more reversed-phase HPLC steps employing hydrophobic RP-HPLC media, (e.g., silica gel having pendant methyl or other aliphatic groups), can be employed to further purify a protein. Some or all of the foregoing purification steps, in various combinations, can be employed to provide a homogeneous recombinant protein.

In some embodiments, recombinant protein produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Application Pub. Nos. 2008/0312425; 2008/0177048; and 2009/0187005.

In certain embodiments, the DLL4 antagonist is a polypeptide that is not an antibody. A variety of methods for identifying and producing non-antibody polypeptides that bind with high affinity to a protein target are known in the art. See, e.g., Skerra, 2007, Curr. Opin. Biotechnol., 18:295-304; Hosse et al., 2006, Protein Science, 15:14-27; Gill et al., 2006, Curr. Opin. Biotechnol., 17:653-658; Nygren, 2008, FEBS J., 275:2668-76; and Skerra, 2008, FEBS J., 275:2677-83. In certain embodiments, phage display technology may be used to produce and/or identify a DLL4 antagonist polypeptide. In certain embodiments, the DLL4 antagonist polypeptide comprises a protein scaffold of a type selected from the group consisting of protein A, protein G, a lipocalin, a fibronectin domain, an ankyrin consensus repeat domain, and thioredoxin.

In certain embodiments, the DLL4 antagonists or antibodies can be used in any one of a number of conjugated (e.g., an immunoconjugate or radioconjugate) or non-conjugated forms. In certain embodiments, the antibodies are used in non-conjugated form to harness the subject's natural defense mechanisms including complement-dependent cytotoxicity (CDC) and/or antibody dependent cellular toxicity (ADCC) to eliminate malignant or cancerous cells.

In certain embodiments, the DLL4 antagonist (e.g., an antibody or polypeptide) is conjugated to a cytotoxic agent. In some embodiments, the cytotoxic agent is a chemotherapeutic agent including, but not limited to, methotrexate, adriamicin, doxorubicin, melphalan, mitomycin C, chlorambucil, daunorubicin or other intercalating agents. In some embodiments, the cytotoxic agent is a enzymatically active toxin of bacterial, fungal, plant, or animal origin, or fragments thereof, including but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, restrictocin, phenomycin, enomycin, and the tricothecenes. In certain embodiments, the cytotoxic agent is a radioactive isotope to produce a radioconjugate or a radioconjugated antibody. A variety of radionuclides are available for the production of radioconjugated antibodies including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re and ²¹²Bi. Conjugates of an antibody and one or more small molecule toxins, such as a calicheamicin, maytansinoids, a trichothene, and CC1065, and the derivatives of these toxins that have toxin activity, can also be used. Conjugates of an antibody and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

Heteroconjugate antibodies are also within the scope of the present invention. Heteroconjugate antibodies are composed of two covalently joined antibodies. Such antibodies have, for example, been proposed to target immune cells to unwanted cells (U.S. Pat. No. 4,676,980). It is contemplated that the antibodies can be prepared in vitro using known methods in synthetic protein chemistry, including those involving crosslinking agents.

### III. POLYNUCLEOTIDES

In certain embodiments, the invention encompasses polynucleotides comprising polynucleotides that encode a polypeptide that specifically binds a human DLL4 or a fragment of such a polypeptide. The term “polynucleotides that encode a polypeptide” encompasses a polynucleotide which includes only coding sequences for the polypeptide as well as a polynucleotide which includes additional coding and/or non-coding sequences. For example, the invention provides a polynucleotide comprising a nucleic acid sequence that encodes an antibody to a human DLL4 or encodes a fragment of such an antibody. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain embodiments, the polynucleotides comprise the coding sequence for the mature polypeptide fused in the same reading frame to a polynucleotide which aids, for example, in expression and secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to produce the mature form of the polypeptide. The polynucleotides can also encode for a proprotein which is the mature protein plus additional 5′ amino acid residues. A mature protein having a prosequence is a proprotein and is an inactive form of the protein. Once the prosequence is cleaved an active mature protein remains.

In certain embodiments the polynucleotides comprise the coding sequence for the mature polypeptide fused in the same reading frame to a marker sequence that allows, for example, for purification and/or identification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. In some embodiments, the marker sequence is a FLAG-tag, a peptide of sequence DYKDDDK (SEQ ID NO:18) which can be used in conjunction with other affinity tags.

The present invention further relates to variants of the hereinabove described polynucleotides encoding, for example, fragments, analogs, and/or derivatives.

In certain embodiments, the present invention provides isolated polynucleotides comprising polynucleotides having a nucleotide sequence at least 80% identical, at least 85% identical, at least 90% identical, at least 95% identical, and in some embodiments, at least 96%, 97%, 98% or 99% identical to a polynucleotide encoding a polypeptide comprising an antibody, or fragment thereof, to human DLL4 described herein.

As used herein, the phrase a polynucleotide having a nucleotide sequence at least, for example, 95% “identical” to a reference nucleotide sequence is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, the polynucleotide variants contain alterations which produce silent substitutions, additions, or deletions, but do not alter the properties or activities of the encoded polypeptide. In some embodiments, polynucleotide variants contain “silent” substitutions due to the degeneracy of the genetic code. Polynucleotide variants can be produced for a variety of reasons, for example, to optimize codon expression for a particular host (e.g., change codons in the human mRNA to those preferred by a bacterial host such as E. coli).

In certain embodiments, the polynucleotides as described herein are isolated. In certain embodiments, the polynucleotides as described herein are substantially pure.

Vectors and cells comprising the polynucleotides described herein are also provided.

### IV. METHODS OF USE AND PHARMACEUTICAL COMPOSITIONS

The present invention provides methods for inhibiting tumor growth using the DLL4 antagonists (e.g., antibodies) described herein. The present invention provides methods of inhibiting growth of a tumor comprising administering a therapeutically effective amount of a DLL4 antagonist to a human subject in need thereof, wherein the tumor comprises a K-ras mutation. In some embodiments, the tumor comprises more than one K-ras mutation. In some embodiments, the K-ras mutation is an activating mutation. In certain embodiments, the K-ras mutation is in codon 12, in codon 13, in codon 59 or in codon 61. In some embodiments, the K-ras mutation in codon 12 is a glycine to cysteine mutation, a glycine to valine mutation, a glycine to aspartic acid mutation, a glycine to alanine mutation, a glycine to arginine mutation, or a glycine to serine mutation. In some embodiments, the K-ras mutation in codon 12 is a glycine to aspartic acid mutation. In other embodiments, the K-ras mutation in codon 12 is a glycine to valine mutation. In some embodiments, the K-ras mutation in codon 13 is a glycine to cysteine mutation, a glycine to valine mutation, a glycine to aspartic acid mutation, a glycine to alanine mutation, a glycine to arginine mutation, or a glycine to serine mutation. In some embodiments, the K-ras mutation in codon 13 is a glycine to aspartic acid mutation. In some embodiments, the K-ras mutation in codon 59 is an alanine to glycine mutation, alanine to valine mutation and alanine to glutamic acid mutation. In other embodiments, the K-ras mutation in codon 61 is a glutamine to leucine mutation, glutamine to proline mutation, glutamine to arginine mutation, and glutamine to histidine mutation. In some embodiments, the K-ras mutation in codon 61 is a glutamine to histidine mutation.

In some embodiments, the tumor comprising a K-ras mutation is substantially non-responsive to at least one EGFR inhibitor. In some embodiments, the EGFR inhibitor is a small molecule compound inhibitor. In some embodiments, the EGFR inhibitor is erlotinib (TARCEVA). In some embodiments, the EGFR inhibitor is gefitinib (IRESSA). In some embodiments, the EGFR inhibitor is an anti-EGFR antibody or antibody fragment. In some embodiments, the anti-EGFR antibody is cetuximab (ERBITUX) or panitumumab (VECTIBIX).

In certain embodiments, the method of inhibiting tumor growth comprises contacting tumor cells with a DLL4 antagonist (e.g., an antibody) in vitro. For example, an immortalized cell line or a cancer cell line that expresses DLL4 on the cell surface is cultured in medium to which is added the antibody or other agent to inhibit tumor cell growth. In some embodiments, tumor cells are isolated from a patient sample (e.g., a tissue biopsy, pleural effusion, or blood sample), and cultured in medium to which is added a DLL4 antagonist to inhibit tumor growth.

In some embodiments, the method of inhibiting tumor growth comprises contacting the tumor or tumor cells with a DLL4 antagonist (e.g., an antibody) in vivo. In certain embodiments, contacting a tumor or tumor cells with a DLL4 antagonist is undertaken in an animal model. For example, DLL4 antagonists are administered to immunocompromised mice (e.g., NOD/SCID mice) that have xenograft tumors expressing DLL4. After administration of DLL4 antagonists, the mice are observed for inhibition of tumor growth. In some embodiments, cancer stem cells are isolated from a patient sample such as, for example, a tissue biopsy, pleural effusion, or blood sample and injected into immunocompromised mice that are then administered a DLL4 antagonist to inhibit tumor growth. In some embodiments, the DLL4 antagonist is administered at the same time or shortly after introduction of tumorigenic cells into the animal to prevent tumor growth. In some embodiments, the DLL4 antagonist is administered as a therapeutic after the tumorigenic cells have grown to a specified size.

The present invention further provides methods of inhibiting growth of a tumor comprising administering a therapeutically effective amount of a DLL4 antagonist as described herein to a human subject in need thereof, wherein the tumor is substantially non-responsive to at least one EGFR inhibitor. In some embodiments, the tumor that is substantially non-responsive to at least one EGFR inhibitor comprises at least one K-ras mutation. In some embodiments, the K-ras mutation is an activating mutation.

In certain embodiments, the method of inhibiting tumor growth comprises administering to a subject a therapeutically effective amount of a DLL4 antagonist. In certain embodiments, the subject is a human. In certain embodiments, the subject has a tumor comprising a K-ras mutation. In certain embodiments, the subject has had a tumor removed. In some embodiments, the DLL4 antagonist is an antibody. In some embodiments, the DLL4 antagonist is a humanized form of antibody 21M18. In some embodiments, the DLL4 antagonist is antibody 21M18 H7L2. In some embodiments, the DLL4 antagonist is antibody 21M18 H9L2.

In certain embodiments, the tumor expresses DLL4 to which the DLL4 antagonist or antibody binds. In certain embodiments, the tumor over-expresses DLL4. In certain embodiments, the tumor expresses a Notch receptor (e.g., Notch1, Notch2, Notch3 and/or Notch4) with which DLL4 interacts.

In certain embodiments, the tumor is a tumor selected from the group consisting of colorectal tumor, pancreatic tumor, lung tumor, ovarian tumor, liver tumor, breast tumor, kidney tumor, prostate tumor, gastrointestinal tumor, melanoma, cervical tumor, bladder tumor, glioblastoma, and head and neck tumor. In some embodiments, the tumor comprising a K-ras mutation is a colorectal tumor, a lung tumor, a liver tumor, a pancreatic tumor, a breast tumor, a prostate tumor, or multiple myeloma. In some embodiments, the tumor is a colorectal tumor. In some embodiments, the tumor is a pancreatic tumor. In some embodiments, the tumor is a lung tumor.

The present invention further provides methods for treating cancer using the DLL4 antagonists described herein. In certain embodiments, the cancer is characterized by cells expressing DLL4 to which the DLL4 antagonist (e.g., antibody) binds. In certain embodiments, the cancer is characterized by cells expressing Notch receptors, wherein the DLL4 antagonist (e.g., an antibody) interferes with DLL4-induced Notch activation and/or signaling. In some embodiments, the DLL4 antagonist binds to DLL4 and inhibits or reduces growth of the cancer. In some embodiments, the DLL4 antagonist binds to DLL4 and inhibits or reduces recurrence of growth of the cancer. In some embodiments, the DLL4 antagonist binds to DLL4, interferes with DLL4/Notch interactions and inhibits or reduces growth of the cancer. In some embodiments, the DLL4 antagonist binds to DLL4, inhibits Notch signaling and inhibits or reduces growth of the cancer. In certain embodiments, the DLL4 antagonist binds to DLL4 and inhibits or reduces angiogenesis. In certain embodiments, the inhibition and/or reduction of angiogenesis inhibits or reduces growth of the cancer.

The present invention provides methods of treating cancer in a human subject, comprising: (a) determining that the subject's cancer comprises a K-ras mutation, and (b) administering to the subject (e.g., a subject in need of treatment) a therapeutically effective amount of a DLL4 antagonist as described herein. In certain embodiments, the subject has a cancerous tumor. In certain embodiments, the subject has had a cancer or tumor removed. In some embodiments, the DLL4 antagonist is an antibody that specifically binds the extracellular domain of human DLL4. In some embodiments, the DLL4 antagonist is antibody 21M18. In some embodiments, the DLL4 antagonist is antibody 21M18 H7L2. In some embodiments, the DLL4 antagonist is antibody 21M18 H9L2.

The present invention further provides methods of treating cancer in a human subject, comprising: (a) selecting a subject for treatment based, at least in part, on the subject having a cancer that comprises a K-ras mutation, and (b) administering to the subject a therapeutically effective amount of a DLL4 antagonist as described herein. In certain embodiments, the subject has a cancerous tumor. In certain embodiments, the subject has had a cancer or tumor removed. In some embodiments, the DLL4 antagonist is an antibody that specifically binds the extracellular domain of human DLL4. In some embodiments, the DLL4 antagonist is antibody 21M18. In some embodiments, the DLL4 antagonist is antibody 21M18 H7L2. In some embodiments, the DLL4 antagonist is antibody 21M18 H9L2.

The present invention further provides methods of treating cancer in a human subject, comprising: (a) identifying a subject that has a cancer comprising a K-ras mutation, and (b) administering to the subject a therapeutically effective amount of a DLL4 antagonist as described herein. In certain embodiments, the subject has a cancerous tumor. In certain embodiments, the subject has had a cancer or tumor removed. In some embodiments, the DLL4 antagonist is an antibody that specifically binds the extracellular domain of human DLL4. In some embodiments, the DLL4 antagonist is antibody 21M18. In some embodiments, the DLL4 antagonist is antibody 21M18 H7L2. In some embodiments, the DLL4 antagonist is antibody 21M18 H9L2.

In some embodiments, the tumor comprising a K-ras mutation is substantially non-responsive to at least one EGFR inhibitor. In some embodiments, the EGFR inhibitor is a small molecule compound inhibitor. In some embodiments, the EGFR inhibitor is erlotinib (TARCEVA). In some embodiments, the EGFR inhibitor is gefitinib (IRESSA). In some embodiments, the EGFR inhibitor is an anti-EGFR antibody or antibody fragment. In some embodiments, the anti-EGFR antibody is cetuximab (ERBITUX) or panitumumab (VECTIBIX).

The present invention further provides methods of treating cancer in a human subject, comprising: (a) determining that the subject's cancer is substantially non-responsive to at least one EGFR inhibitor, and (b) administering to the subject a therapeutically effective amount of a DLL4 antagonist as described herein. In some embodiments, the cancer comprises at least one K-ras mutation. In some embodiments, the K-ras mutation is an activating mutation. In some embodiments, the EGFR inhibitor is a small molecule compound inhibitor. In some embodiments, the EGFR inhibitor is erlotinib. In some embodiments, the EGFR inhibitor is gefitinib. In some embodiments, the EGFR inhibitor is an anti-EGFR antibody. In some embodiments, the anti-EGFR antibody is cetuximab or panitumumab. In certain embodiments, the subject has had a cancer or tumor removed. In some embodiments, the DLL4 antagonist is an antibody that specifically binds the extracellular domain of human DLL4. In some embodiments, the DLL4 antagonist is antibody 21M18. In some embodiments, the DLL4 antagonist is antibody 21M18 H7L2. In some embodiments, the DLL4 antagonist is antibody 21M18 H9L2.

The present invention further provides methods of treating cancer in a human subject, comprising: (a) selecting a subject for treatment based, at least in part, on the subject having a cancer that is substantially non-responsive to at least one EGFR inhibitor, and (b) administering to the subject a therapeutically effective amount of a DLL4 antagonist as described herein. In some embodiments, the cancer comprises at least one K-ras mutation. In some embodiments, the K-ras mutation is an activating mutation. In some embodiments, the EGFR inhibitor is a small molecule compound inhibitor. In some embodiments, the EGFR inhibitor is erlotinib. In some embodiments, the EGFR inhibitor is gefitinib. In some embodiments, the EGFR inhibitor is an anti-EGFR antibody. In some embodiments, the anti-EGFR antibody is cetuximab or panitumumab. In certain embodiments, the subject has had a cancer or tumor removed. In some embodiments, the DLL4 antagonist is an antibody that specifically binds the extracellular domain of human DLL4. In some embodiments, the DLL4 antagonist is antibody 21M18. In some embodiments, the DLL4 antagonist is antibody 21M18 H7L2. In some embodiments, the DLL4 antagonist is antibody 21M18 H9L2.

The present invention further provides methods of treating cancer in a human subject, comprising: (a) identifying a subject that has a cancer that is substantially non-responsive to at least one EGFR inhibitor, and (b) administering to the subject a therapeutically effective amount of a DLL4 antagonist as described herein. In some embodiments, the cancer comprises at least one K-ras mutation. In some embodiments, the K-ras mutation is an activating mutation. In some embodiments, the EGFR inhibitor is a small molecule compound inhibitor. In some embodiments, the EGFR inhibitor is erlotinib. In some embodiments, the EGFR inhibitor is gefitinib. In some embodiments, the EGFR inhibitor is an anti-EGFR antibody. In some embodiments, the anti-EGFR antibody is cetuximab or panitumumab. In certain embodiments, the subject has had a cancer or tumor removed. In some embodiments, the DLL4 antagonist is an antibody that specifically binds the extracellular domain of human DLL4. In some embodiments, the DLL4 antagonist is antibody 21M18. In some embodiments, the DLL4 antagonist is antibody 21M18 H7L2. In some embodiments, the DLL4 antagonist is antibody 21M18 H9L2.

The present invention further provides methods of selecting a human subject for treatment with a DLL4 antagonist. In some embodiments, the methods comprise determining if the subject has (a) a cancer comprising a K-ras mutation or (b) a cancer that is substantially non-responsive to at least one EGFR inhibitor, wherein if the subject has (a) and/or (b), the subject is selected for treatment with a DLL4 antagonist as described herein. In some embodiments, the DLL4 antagonist is an antibody that specifically binds the extracellular domain of human DLL4. In some embodiments, the K-ras mutation is an activating mutation. In some embodiments, the EGFR inhibitor is a small molecule compound inhibitor. In some embodiments, the EGFR inhibitor is erlotinib. In some embodiments, the EGFR inhibitor is gefitinib. In some embodiments, the EGFR inhibitor is an anti-EGFR antibody. In some embodiments, the anti-EGFR antibody is cetuximab or panitumumab. In certain embodiments, the subject has had a cancer or tumor removed. In some embodiments, the DLL4 antagonist is an antibody that specifically binds the extracellular domain of human DLL4. In some embodiments, the DLL4 antagonist is antibody 21M18. In some embodiments, the DLL4 antagonist is antibody 21M18 H7L2. In some embodiments, the DLL4 antagonist is antibody 21M18 H9L2.

In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, bladder cancer, glioblastoma, and head and neck cancer. In certain embodiments, the cancer is pancreatic cancer. In certain embodiments, the cancer is colorectal cancer. In certain embodiments, the cancer is breast cancer. In certain embodiments, the cancer is prostate cancer. In certain embodiments, the cancer is lung cancer.

The sequence of wild-type human K-Ras is known in the art, (e.g. Accession No. NP_203524). Methods for determining whether a tumor or cancer comprises a K-ras mutation can be undertaken by assessing the nucleotide sequence encoding the K-ras protein, by assessing the amino acid sequence of the K-ras protein, or by assessing the characteristics of a putative K-ras mutant protein.

Methods for detecting a mutation in a K-ras nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples may be evaluated for K-ras mutations by real-time PCR. In real-time PCR, fluorescent probes specific for the most common mutations (e.g., mutation in codons 12, 13, 59 and/or 61) are used. When a mutation is present, the probe binds and fluorescence is detected. In some embodiments, K-ras mutations may be identified using a direct sequencing method of specific regions (e.g., exon 2 and/or exon 3) in the K-ras gene. This technique will identify all possible mutations in the region sequenced.

Methods for detecting a mutation in a K-ras protein are known by those of skill in the art. These methods include, but are not limited to, detection of a K-ras mutant using a binding agent (e.g., an antibody) specific for the mutant protein, protein electrophoresis and Western blotting, and direct peptide sequencing.

Methods for determining whether a tumor or cancer comprises a K-ras mutation can use a variety of samples. In some embodiments, the sample is taken from a subject having a tumor or cancer. In some embodiments, the sample is taken from a subject having a cancer or tumor that is substantially non-responsive to at least one EGFR inhibitor. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is processed to a cell lysate. In some embodiments, the sample is processed to DNA or RNA.

The invention also provides a method of inhibiting Notch signaling in a cell comprising contacting the cell with an effective amount of a DLL4 antagonist. In certain embodiments, the cell is a tumor cell. In some embodiments, the tumor cell comprises at least one K-ras mutation. In some embodiments, the tumor cell is substantially non-responsive to at least one EGFR inhibitor. In certain embodiments, the method is an in vivo method wherein the step of contacting the cell with the DLL4 antagonist comprises administering a therapeutically effective amount of the DLL4 antagonist to the subject. In some embodiments, the method is an in vitro or ex vivo method. In certain embodiments, the DLL4 antagonist interferes with Notch signaling. In certain embodiments, the DLL4 antagonist interferes with a DLL4/Notch interaction. In certain embodiments, the Notch signaling is signaling by Notch1, Notch2, Notch3, and/or Notch4. In some embodiments, the DLL4 antagonist is an antibody. In some embodiments, the DLL4 antagonist is antibody 21M18, 21M18 H7L2 or 21M18 H9L2.

In addition, the invention provides a method of reducing the tumorigenicity of a tumor in a subject, comprising administering a therapeutically effective amount of a DLL4 antagonist to the subject. In some embodiments, the tumor comprises at least one K-ras mutation. In certain embodiments, the tumor comprises cancer stem cells. In some embodiments, the cancer stem cells comprise at least one K-ras mutation. In some embodiments, the cancer stem cells are substantially non-responsive to at least one EGFR inhibitor. In certain embodiments, the frequency of cancer stem cells in the tumor is reduced by administration of the DLL4 antagonist. Thus, the invention also provides a method of reducing the frequency of cancer stem cells in a tumor comprising at least one K-ras mutation, comprising contacting the tumor with an effective amount of a DLL4 antagonist (e.g., an anti-DLL4 antibody).

The invention also provides a method of treating a disease or disorder in a subject, wherein the disease or disorder is characterized by an increased level of stem cells and/or progenitor cells. In some embodiments, the stem cells and/or progenitor cells comprise at least one K-ras mutation. In some embodiments, the treatment methods comprise administering a therapeutically effective amount of the DLL4 antagonist, polypeptide, or antibody to the subject.

The present invention further provides pharmaceutical compositions comprising one or more of the DLL4 antagonists described herein. In certain embodiments, the pharmaceutical compositions further comprise a pharmaceutically acceptable vehicle. These pharmaceutical compositions find use in inhibiting tumor growth and treating cancer in a subject (e.g., a human patient).

In certain embodiments, formulations are prepared for storage and use by combining a purified antibody or agent of the present invention with a pharmaceutically acceptable vehicle (e.g., a carrier or excipient). Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 21st Edition, University of the Sciences in Philadelphia, 2005).

In certain embodiments, the anti-DLL4 antagonist or antibody can be prepared for use at a concentration of 10 mg/mL in a solution of 50 mM histidine, 100 mM sodium chloride, 45 mM sucrose, and 0.01% (w/v) Polysorbate 20, and the pH adjusted to 6.0.

The pharmaceutical compositions of the present invention can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present invention, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The antibodies or agents described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 21st Edition, University of the Sciences in Philadelphia, 2005.

In certain embodiments, pharmaceutical formulations include DLL4 antagonists (e.g., an antibody) of the present invention complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations can be produced. Suitable examples of sustained-release preparations include semi-permeable matrices of solid hydrophobic polymers containing the DLL4 antagonist (e.g., an antibody), where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

In certain embodiments, in addition to administering a DLL4 antagonist (e.g., an antibody), the method or treatment further comprises administering at least one additional therapeutic agent. An additional therapeutic agent can be administered prior to, concurrently with, and/or subsequently to, administration of the DLL4 antagonist. Pharmaceutical compositions comprising the DLL4 antagonist and the additional therapeutic agent(s) are also provided. In some embodiments, the at least one additional therapeutic agent comprises 1, 2, 3, or more additional therapeutic agents.

Combination therapy with at least two therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects. Combination therapy may decrease the likelihood that resistant cancer cells will develop.

It will be appreciated that the combination of a DLL4 antagonist and an additional therapeutic agent may be administered in any order or concurrently. In some embodiments, the DLL4 antagonists will be administered to patients that have previously undergone treatment with a second therapeutic agent. In certain other embodiments, the DLL4 antagonist and a second therapeutic agent will be administered substantially simultaneously or concurrently. For example, a subject may be given the DLL4 antagonist (e.g., an antibody) while undergoing a course of treatment with a second therapeutic agent (e.g., chemotherapy). In certain embodiments, the DLL4 antagonist will be administered within 1 year of the treatment with a second therapeutic agent. In certain alternative embodiments, the DLL4 antagonist will be administered within 10, 8, 6, 4, or 2 months of any treatment with a second therapeutic agent. In certain other embodiments, the DLL4 antagonist will be administered within 4, 3, 2, or 1 weeks of any treatment with a second therapeutic agent. In some embodiments, the DLL4 antagonist will be administered within 5, 4, 3, 2, or 1 days of any treatment with a second therapeutic agent. It will further be appreciated that the two (or more) agents or treatment may be administered to the subject within a matter of hours or minutes (i.e., substantially simultaneously).

Useful classes of therapeutic agents include, for example, antitubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an antimetabolite, an antimitotic, a topoisomerase inhibitor, or an angiogenesis inhibitor.

Therapeutic agents that may be administered in combination with the DLL4 antagonists include chemotherapeutic agents. Thus, in some embodiments, the method or treatment involves the combined administration of a DLL4 antagonist or antibody of the present invention and a chemotherapeutic agent or cocktail of multiple different chemotherapeutic agents. Treatment with an antibody can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992).

Chemotherapeutic agents useful in the instant invention include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitor RFS 2000; difluoromethylornithine (DMFO); retinoic acid; esperamicins; capecitabine; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCL, daunorubicin citrate, mitoxantrone HCl, actinomycin D, etoposide, topotecan HCl, teniposide (VM-26), and irinotecan. In certain embodiments, the second therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, THIOGUANINE, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the second therapeutic agent is gemcitabine.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, binblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In certain embodiments, where the chemotherapeutic agent administered in combination with the DLL4 antagonist is an anti-mitotic agent, the cancer or tumor being treated is breast cancer or a breast tumor.

In certain embodiments, the treatment involves the combined administration of a DLL4 antagonist (e.g. an antibody) of the present invention and radiation therapy. Treatment with the DLL4 antagonist can occur prior to, concurrently with, or subsequent to administration of radiation therapy. Dosing schedules for such radiation therapy can be determined by the skilled medical practitioner.

In some embodiments, a second therapeutic agent comprises an antibody. Thus, treatment can involve the combined administration of a DLL4 antagonist (e.g. an antibody) of the present invention with other antibodies against additional tumor-associated antigens including, but not limited to, antibodies that bind to ErbB2, HER2, Jagged, Notch and/or VEGF. Exemplary anti-Notch antibodies, are described, for example, in U.S. Patent Application Publication No. 2008/0131434. In certain embodiments, a second therapeutic agent is an antibody that is an angiogenesis inhibitor (e.g., an anti-VEGF antibody). In certain embodiments, a second therapeutic agent is bevacizumab (AVASTIN), or trastuzumab (HERCEPTIN). In some embodiments, the second therapeutic agent is not an anti-EGFR antibody. In some embodiments, the second therapeutic agent is not panitumumab (VECTIBIX) or cetuximab (ERBITUX). Combined administration can include co-administration, either in a single pharmaceutical formulation or using separate formulations, or consecutive administration in either order but generally within a time period such that all active agents can exert their biological activities simultaneously.

Furthermore, treatment with the DLL4 antagonists described herein can include combination treatment with one or more cytokines (e.g., lymphokines, interleukins, tumor necrosis factors, and/or growth factors) or can be accompanied by surgical removal of tumors, cancer cells or any other therapy deemed necessary by a treating physician.

For the treatment of the disease, the appropriate dosage of an DLL4 antagonist (e.g., an antibody) of the present invention depends on the type of disease to be treated, the severity and course of the disease, the responsiveness of the disease, whether the DLL4 antagonist or antibody is administered for therapeutic or preventative purposes, previous therapy, the patient's clinical history, and so on, all at the discretion of the treating physician. The DLL4 antagonist or antibody can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual antibody or agent. The administering physician can easily determine optimum dosages, dosing methodologies and repetition rates. In certain embodiments, dosage is from 0.01 μg to 100 mg per kg of body weight, and can be given once or more daily, weekly, monthly or yearly. In certain embodiments, the DLL4 antagonist or antibody is given once every two weeks or once every three weeks. In certain embodiments, the dosage of the DLL4 antagonist or antibody is from about 0.1 mg to about 20 mg per kg of body weight. The treating physician can estimate repetition rates for dosing based on measured residence times and concentrations of the drug in bodily fluids or tissues.

### EXAMPLES

### Example 1

### Evaluation of Tumors for K-Ras Gene Mutations

A large collection of xenografts derived from primary patient tumors including colon cancer have been established. Genomic DNA samples were isolated from primary and passaged tumors using a Genomic DNA Extraction Kit (Bioneer Inc., Alameda Calif.) following the manufacturers' instructions. The quality of the isolated DNA was checked by visualizing the DNA samples on a 1% agarose gel or a 0.8% E-Gel (Invitrogen Corporation, Carlsbad, Calif.). The DNA was confirmed to be intact by the presence of an approximately 20 kb size band with little or no visible degradation. The purified genomic DNA samples were sent to SeqWright Technologies, (Houston Tex.) for nucleotide sequence analysis. The K-ras gene was obtained by amplifying genomic DNA samples with the Repli-G Mini Kit (Qiagen, Valencia Calif.) followed by PCR amplification and purification. The nucleotide sequence of the K-ras gene for each tumor was obtained using an ABI 3730xL DNA Sequencer (Applied Biosystems, Foster City, Calif.).

Of the eight colon tumors evaluated, 3 had a wild type K-ras gene (C8, C27 and C40) as compared to the human K-ras sequence (see e.g. Accession No. NP_203524). However, C27 is not considered a true wild type K-ras tumor because it has a mutation in the downstream B-raf gene, which can confer constitutive activation of the pathway. Mutations in K-ras and B-raf appear to be mutually exclusive. Two tumors had a mutation in codon 12, one a glycine to aspartic acid mutation (C4) and one a glycine to valine mutation (C9). Two tumors had a mutation in codon 13, both were glycine to aspartic acid mutations (C6 and C12). One tumor had a mutation in codon 61, an aspartic acid to histidine mutation (C22). The K-ras gene status of the tumors is summarized in Table 1. The K-ras mutations identified in tumors C4, C6, C9, C12 and C22 are known activating mutations.

TABLE 1

| | Tumor | | | | | | | |
|---|---|---|---|---|---|---|---|---|
| | C4 | C6 | C8 | C9 | C12 | C22 | C27 | C40 |
| K-ras | G12D | G13D | WT* | G12V | G13D | Q61H | B-raf^{MT} | WT |

WT* = wild-type K-ras gene

### Example 2

### Evaluation of Anti-Tumor Activity of Anti-EGFR Antibody Alone and Anti-DLL4 Antibody, Alone or in Combination with a Chemotherapeutic Agent, in Colon Tumor Xenograft Models

NOD/SCID mice were purchased from Harlan Laboratories (Indianapolis, Ind.) and maintained under specific pathogen-free conditions and provided with sterile food and water ad libitum. The animals were housed in a U.S. Department of Agriculture-registered facility in accordance with NIH guidelines for the care and use of laboratory animals. The mice were allowed to acclimate for several days prior to the start of each study.

In general, tumor cells from a patient sample that have been passed as a xenograft in mice were prepared for injection into experimental animals. Tumor tissue was removed under sterile conditions, cut up into small pieces, minced completely using sterile blades, and single cell suspensions obtained by enzymatic digestion and mechanical disruption. Specifically, tumor pieces were mixed with ultra-pure collagenase III in culture medium and incubated at 37° C. for 1-4 hours. Digested cells were filtered through nylon mesh and washed in Hank's buffered saline solution (HBSS) containing 2% heat-inactivated calf serum and 25 mM HEPES (pH 7.4).

Dissociated cells (10,000 cells) were injected subcutaneously into the flanks of 6-8 week old NOD/SCID mice. Tumors were allowed to grow until they were approximately 100-150 mm³. The animals were randomized (n=10 per group) and treated with a control antibody (anti-lyzoyme antibody LZ-1), anti-EGFR antibody, anti-DLL4 antibody, irinotecan or a combination of anti-DLL4 antibody plus irinotecan. The “anti-DLL4 antibody” was a 1:1 mixture of (i) anti-human DLL4 antibody 21M18 H7L2 and (ii) anti-mouse DLL4 antibody 21R30. The anti-EGFR antibody was cetuximab. Antibodies were dosed at 10 mg/kg once a week and irinotecan was dosed at 7.5 mg/kg twice per week. The 10 mg/kg dose of the anti-DLL4 antibody refers to the antibody mixture. Both antibodies and chemotherapeutic agents were administered intraperitoneally. Tumor growth was measured weekly with electronic calipers.

Anti-EGFR antibody (cetuximab) inhibited tumor growth in the two wild-type K-ras tumors, C8 (FIG. 1A) and C40 (FIG. 1B). The anti-EGFR antibody was observed to have no effect on tumor growth in four of five K-ras mutant tumors, C4 (FIG. 1C), C6 (FIG. 1D), C9 (FIG. 1E), C12 (FIG. 1F) and C22 (FIG. 1G). Thus, the majority of K-ras mutant tumors were non-responsive to anti-EGFR antibodies. Additional studies showed that anti-EGFR antibodies in combination with a chemotherapeutic agent, irinotecan, had very similar results. For example, in the wild-type K-ras tumor C8 a combination of anti-EGFR antibody plus irinotecan did not inhibit tumor growth any better than either agent alone (FIG. 1H). In the K-ras mutant tumor C12, the anti-EGFR antibody alone did not reduce tumor growth as compared to the control antibody. A combination of anti-EGFR antibody plus irinotecan had only a slight reduction in tumor growth, but appeared to substantially hinder the anti-tumor effect of irinotecan alone (FIG. 1I). These findings parallel clinical studies that have demonstrated little to no efficacy of anti-EGFR antibodies in treatment of patients with colon cancers comprising K-ras mutations.

Anti-DLL4 antibody (i.e., the 1:1 mixture described above) inhibited tumor growth in the two wild-type K-ras tumors, C8 (FIG. 2A) and C40 (FIG. 2B) without concurrent treatment with a chemotherapeutic agent. Likewise, the anti-DLL4 antibody even in the absence of an additional chemotherapeutic agent was observed to reduce tumor growth in three of five K-ras mutant tumors as compared to the control antibody, C6 (FIG. 2D), C9 (FIG. 2E) and C12 (FIG. 2F). Surprisingly, anti-DLL4 antibody in combination with irinotecan inhibited tumor growth in seven of eight tumors tested, and importantly, in all five of the K-ras mutant tumors (FIGS. 2A-G). The data is summarized in Table 2.

TABLE 2

| | Tumor Volume Reduction, % of Control Ab | | | |
|---|---|---|---|---|
| | | | | Anti-DLL4 + |
| | Anti-DLL4 | Anti-EGFR | Irinotecan | Irinotecan |
| K-ras^{WT} | | | | |
| C8 | 34 ± 9* | 47 ± 9* | 48 ± 8* | 88 ± 2*, ** |
| C40 | 31 ± 4* | 54 ± 8* | 38 ± 8* | 79 ± 3*, ** |
| K-ras^{MT} | | | | |
| C4 | 19 ± 12 | 18 ± 10 | 58 ± 5* | 87 ± 2*, ** |
| C6 | 45 ± 11* | 75 ± 3* | 57 ± 8* | 88 ± 1*, ** |
| C9 | 34 ± 10* | −31 ± 17 | 31 ± 10 | 64 ± 9*, ** |
| C12 | 48 ± 5* | 2 ± 8 | 49 ± 3* | 75 ± 2*, ** |
| C22 | 4 ± 5 | −14 ± 16 | 54 ± 5* | 90 ± 2*, ** |

Data is expressed as mean +/− SEM
*= p < 0.05 vs. control Ab
** = p < 0.01 vs. single agents

### Example 3

### Evaluation of Anti-DLL4 Antibody, Alone or in Combination with a Chemotherapeutic Agent, in a Colon Tumor Xenograft Model for Reduction of Cancer Stem Cell Frequency

The ability of anti-DLL4 antibodies alone, or in combination with irinotecan, to reduce the frequency of cancer stem cells (CSCs) in a K-ras mutant tumor was determined in a limiting dilution assay (LDA). Dissociated C9 colon tumor cells (10,000 cells) were injected subcutaneously into the flanks of 6-8 week old NOD/SCID mice. Tumors were allowed to grow until they were approximately 100-150 mm³. The animals were randomized (n=10 per group) and treated with a control antibody (anti-lysozyme antibody LZ-1), anti-DLL4 antibody, irinotecan or a combination of anti-DLL4 antibody plus irinotecan. The anti-DLL4 antibody was a 1:1 mixture of anti-human DDL4 antibody and anti-mouse DLL4 antibody as described above. Antibodies were dosed at 10 mg/kg once a week and irinotecan was dosed at 7.5 mg/kg twice per week. Both antibodies and chemotherapeutic agents were administered intraperitoneally. Tumor growth was measured weekly with electronic calipers. At the end of the experiment, tumors were harvested, depleted of stromal cells, and the human tumor cells were serially transplanted into a set of mice. Tumors were allowed to grow untreated for 62 days. The tumor take rate was used to calculate the CSC frequency.

As shown in FIG. 3, the CSC frequency in the group treated with the control antibody was 1:149. Treatment with anti-DLL4 antibody reduced CSC frequency to 1:299, approximately a two-fold reduction compared to the control antibody. Treatment with irinotecan alone did not reduce CSC frequency, in fact a slight increase in the CSC frequency (1:105) was observed. Surprisingly, treatment with the combination of anti-DLL4+irinotecan demonstrated a greater reduction in CSC frequency than with anti-DLL4 antibody alone and despite the fact that irinotecan alone had no effect or actually slightly increased CSC frequency. The combination of anti-DLL4 antibody and irinotecan reduced CSC frequency to 1:540, almost a four-fold reduction compared to the control antibody, and almost a two-fold further reduction as compared to anti-DLL4 antibody alone.

### Example 4

### Evaluation of Anti-Tumor Activity of Anti-DLL4 Antibody in Combination with a Chemotherapeutic Agent in a Colon Tumor Recurrence Xenograft Model

Dissociated C9 colon tumor cells (10,000 cells) were injected subcutaneously into the flanks of 6-8 week old NOD/SCID mice (n=10 per group). Starting 2 days after injection, mice were treated with irinotecan alone (Δ) or anti-human DLL4 antibody (21M18 H7L2) plus irinotecan (▾). Treatment in both groups was discontinued on the indicated day and tumor growth was monitored for an additional period of time. Anti-human DLL4 antibody was dosed at 10 mg/kg twice a week and irinotecan was dosed at 7.5 mg/kg once a week. Both antibodies and chemotherapeutic agents were administered intraperitoneally. Tumor growth was measured with electronic calipers at the indicated time points.

As shown in FIG. 4, after cessation of treatment, tumor growth progressed in the group previously treated with irinotecan alone. In contrast, the group previously treated with the combination of anti-human DLL4 antibody plus irinotecan exhibited no further tumor growth.

### Example 5

### Evaluation of Anti-Tumor Activity of Anti-DLL4 Antibody after Treatment with a Chemotherapeutic Agent in a Pancreatic Tumor Recurrence Xenograft Model

The PN8 pancreatic tumor was determined to have a K-ras mutation, a glycine to aspartic acid mutation at codon 12, that is a known activating mutation. Anti-DLL4 antibodies were tested for efficacy in this xenograft tumor model. Dissociated PN8 pancreatic tumor cells (10,000 cells) were injected subcutaneously into the flanks of 6-8 week old NOD/SCID mice. Tumors were allowed to grow for 28 days until they reached an average volume of 173 mm³. The mice were randomized (n=10 per group) and treated with gemcitabine at 100 mg/kg once a week for 4 weeks. On day 53, the gemcitabine treatments were stopped and antibody treatments initiated. Mice were treated with control antibody (), anti-mouse DLL4 antibody (21R30) (▾), anti-human DLL4 antibody (21M18 H7L2) (▴), or a combination of anti-mouse DLL4 antibody and anti-human DLL4 antibody (21R30+21M18 H7L2) (□). Antibodies were dosed at 10 mg/kg once a week. Both antibodies and chemotherapeutic agents were administered intraperitoneally. Tumor growth was measured with electronic calipers at the indicated time points.

As shown in FIG. 5, treatment with anti-mouse DLL4 antibody, which blocks DLL4 in the mouse stroma and vascular cells, was found to modestly delay tumor recurrence after chemotherapy as compared to control antibody. Treatment with anti-human DLL4 antibody, which blocks DLL4 in the tumor cells, had a more substantial effect in inhibiting and/or delaying tumor re-growth. Importantly, the combination of the two DLL4 antibodies, one blocking DLL4 at the stroma and one blocking DLL4 on the tumor, was more effective than either alone and appeared to completely block tumor recurrence.

It is understood that the examples and embodiments described herein are for illustrative purposes only and that various modifications or changes in light thereof will be suggested to persons skilled in the art and are to be included within the spirit and purview of this application.

All publications, patents, and patent applications cited herein are hereby incorporated by reference in their entirety for all purposes to the same extent as if each individual publication, patent or patent application were specifically and individually indicated to be so incorporated by reference.

SEQUENCES

SEQ ID NO: 1 Heavy chain CDR1
TAYYIH
SEQ ID NO: 2 Heavy chain CDR2, H2
YISCYNGATNYNQKFKG
SEQ ID NO: 3 Heavy chain CDR2, H7
YISSYNGATNYNQKFKG
SEQ ID NO: 4 Heavy chain CDR2 H9
YISVYNGATNYNQKFKG
SEQ ID NO: 5 Heavy chain CDR3
RDYDYDVGMDY
SEQ ID NO: 6 Heavy chain variable region without
signal sequence, H7
QVQLVQSGAEVKKPGASVKISCKASGYSFTAYYIHWVKQAPGQGLEWIGY
ISSYNGATNYNQKFKGRVTFTTDTSTSTAYMELRSLRSDDTAVYYCARDY
DYDVGMDYWGQGTLVTVSS
SEQ ID NO: 7 Light chain CDR1
RASESVDNYGISFMK
SEQ ID NO: 8 Light chain CDR2
AASNQGS
SEQ ID NO: 9 Light chain CDR3
QQSKEVPWTFGG
SEQ ID NO: 10 Light chain variable region without
signal sequence
DIVMTQSPDSLAVSLGERATISCRASESVDNYGISFMKWFQQKPGQPPKL
LIYAASNQGSGVPDRFSGSGSGTDFTLTISSLQAEDVAVYYCQQSKEVPW
TFGGGTKVEIK
SEQ ID NO: 11 Amino acids 27-217 of Human DLL4
Extracellular Domain (without putative signal
sequence)
SGVFQLQLQEFINERGVLASGRPCEPGCRTFFRVCLKHFQAVVSPGPCTF
GTVSTPVLGTNSFAVRDDSSGGGRNPLQLPFNFTWPGTFSLIIEAWHAPG
DDLRPEALPPDALISKIAIQGSLAVGQNWLLDEQTSTLTRLRYSYRVICS
DNYYGDNCSRLCKKRNDHFGHYVCQPDGNLSCLPGWTGEYC
SEQ ID NO: 12 Heavy chain variable region without
signal sequence, H2
QVQLVQSGAEVKKPGASVKISCKASGYSFTAYYIHWVKQAPGQGLEWIGY
ISCYNGATNYNQKFKGRVTFTTDTSTSTAYMELRSLRSDDTAVYYCARDY
DYDVGMDYWGQGTLVTVSS
SEQ ID NO: 13 Heavy chain variable region without
signal sequence, H9
QVQLVQSGAEVKKPGASVKISCKASGYSFTAYYIHWVKQAPGQGLEWIGY
ISVYNGATNYNQKFKGRVTFTTDTSTSTAYMELRSLRSDDTAVYYCARDY
DYDVGMDYWGQGTLVTVSS
SEQ ID NO: 14 Human K-ras amino acid sequence
MTEYKLVVVGAGGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKIRQ
RVEDAFYTLVREIRQYRLKKISKEEKTPGCVKIKKCIIM
SEQ ID NO: 15 Human DLL4 Extracellular Domain
(without putative signal sequence)
SGVFQLQLQEFINERGVLASGRPCEPGCRTFFRVCLKHFQAVVSPGPCTF
GTVSTPVLGTNSFAVRDDSSGGGRNPLQLPFNFTWPGTFSLIIEAWHAPG
DDLRPEALPPDALISKIAIQGSLAVGQNWLLDEQTSTLTRLRYSYRVICS
DNYYGDNCSRLCKKRNDHFGHYVCQPDGNLSCLPGWTGEYCQQPICLSGC
HEQNGYCSKPAECLCRPGWQGRLCNECIPHNGCRHGTCSTPWQCTCDEGW
GGLFCDQDLNYCTHHSPCKNGATCSNSGQRSYTCTCRPGYTGVDCELELS
ECDSNPCRNGGSCKDQEDGYHCLCPPGYYGLHCEHSTLSCADSPCFNGGS
CRERNQGANYACECPPNFTGSNCEKKVDRCTSNPCANGGQCLNRGPSRMC
RCRPGFTGTYCELHVSDCARNPCAHGGTCHDLENGLMCTCPAGFSGRRCE
VRTSIDACASSPCFNRATCYTDLSTDTFVCNCPYGFVGSRCEFPVG
SEQ ID NO: 16 Human DLL4 N-Terminal Region (with
putative signal sequence)
MAAASRSASGTNALLLLVALWQQRAAGSGVFQLQLQEFINERGVLASGRP
CEPGCRTFFRVCLKHFQAVVSPGPCTFGTVSTPVLGTNSFAVRDDSSGGG
RNPLQLPFNFTWPGTFSLIIEAWHAPGDDLRPEALPPDALISKIAIQGSL
AVGQN
SEQ ID NO: 17 Human DLL4 DSL Region
WLLDEQTSTLTRLRYSYRVICSDNYYGDNCSRLCKKRNDHFGHYVCQPDG
NLSCLPGYNTGEYC
SEQ ID NO: 18 FLAG tag
DYKDDDK

1-91. (canceled)

92. A method of treating cancer in a human subject comprising administering a therapeutically effective amount of a delta-like ligand 4 (DLL4) antagonist and at least one additional therapeutic agent to the subject, wherein the cancer is substantially non-responsive to at least one epithelial growth factor receptor (EGFR) inhibitor, and wherein the DLL4 antagonist is an antibody that specifically binds the extracellular domain of human DLL4.

93. The method of claim 92, wherein the antibody specifically binds an epitope comprising amino acids within the N-terminal region of the extracellular domain of human DLL4 (SEQ ID NO:16).

94. The method of claim 92, wherein the antibody comprises:
(a) a heavy chain CDR1 comprising TAYYIH (SEQ ID NO:1), a heavy chain CDR2 comprising YISCYNGATNYNQKFKG (SEQ ID NO:2), YISSYNGATNYNQKFKG (SEQ ID NO:3), or YISVYNGATNYNQKFKG (SEQ ID NO:4), and a heavy chain CDR3 comprising RDYDYDVGMDY (SEQ ID NO:5); and
(b) a light chain CDR1 comprising RASESVDNYGISFMK (SEQ ID NO:7), a light chain CDR2 comprising AASNQGS (SEQ ID NO:8), and a light chain CDR3 comprising QQSKEVPWTFGG (SEQ ID NO:9).

95. The method of claim 94, wherein the antibody comprises:
(a) a heavy chain CDR1 comprising TAYYIH (SEQ ID NO:1), a heavy chain CDR2 comprising YISSYNGATNYNQKFKG (SEQ ID NO:3), and a heavy chain CDR3 comprising RDYDYDVGMDY (SEQ ID NO:5); and
(b) a light chain CDR1 comprising RASESVDNYGISFMK (SEQ ID NO:7), a light chain CDR2 comprising AASNQGS (SEQ ID NO:8), and a light chain CDR3 comprising QQSKEVPWTFGG (SEQ ID NO:9).

96. The method of claim 94, wherein the antibody comprises:
(a) a heavy chain variable region having at least about 90% sequence identity to SEQ ID NO:6, SEQ ID NO:12 or SEQ ID NO:13; and
(b) a light chain variable region having at least about 90% sequence identity to SEQ ID NO:10.

96. The method of claim 96, wherein the antibody comprises:
(a) a heavy chain variable region comprising SEQ ID NO:6; and
(b) a light chain variable region comprising SEQ ID NO:10.

97. The method of claim 92, wherein the antibody is a monoclonal antibody, a chimeric antibody, a bispecific antibody, a humanized antibody, a human antibody, or an antibody fragment.

98. The method of claim 92, wherein the antibody competes for specific binding to human DLL4 with an antibody encoded by the plasmid deposited with ATCC having deposit no. PTA-8425.

99. The method of claim 92, wherein the additional therapeutic agent is a chemotherapeutic agent.

100. The method of claim 104, wherein the chemotherapeutic agent is selected from the group consisting of: a topoisomerase inhibitor, an anti-metabolite, an anti-mitotic agent, and an alkylating agent.

101. The method of claim 92, wherein the additional therapeutic agent is an angiogenesis inhibitor.

102. The method of claim 92, wherein the cancer is selected from the group consisting of a colorectal cancer, a lung cancer, a liver cancer, and a pancreatic cancer.

103. The method of claim 92, wherein the EGFR inhibitor is a small molecule compound or an antibody.

104. The method of claim 92, wherein the EGFR inhibitor is an anti-EGFR antibody.

105. The method of claim 104, wherein the anti-EGFR antibody is cetuximab or panitumumab.

106. The method of claim 92, wherein the EGFR inhibitor is erlotinib or gefitinib.

107. A method of selecting a human subject for treatment with a delta-like ligand 4 (DLL4) antagonist, comprising determining if the subject has:
(a) a cancer comprising a K-ras mutation or
(b) a cancer that is substantially non-responsive to at least one EGFR inhibitor,
wherein if the subject has (a) and/or (b), the subject is selected for treatment with a DLL4 antagonist.
